(19) United States
(12) Patent Application Publication
(10) Pub. No.: US 2016/0280851 A1
(43) Pub. Date: Sep. 29, 2016

(54) IMINOETHER COMPOUND, POLYESTER RESIN COMPOSITION, METHOD FOR PRODUCING CARBOXYLIC ACID ESTER, POLYESTER FILM, BACK SHEET FOR SOLAR CELL MODULE, AND SOLAR CELL MODULE

(71) Applicant: FUJIFILM Corporation, Tokyo (JP)

(72) Inventors: Shigeki UEHIRA, Fujinomiya-shi (JP); Makoto FUKUDA, Fujinomiya-shi (JP); Michihiro OGAWA, Fujinomiya-shi (JP); Masatoshi MIZUMURA, Fujinomiya-shi (JP)

(73) Assignee: FUJIFILM Corporation, Tokyo (JP)

(21) Appl. No.: 15/176,835

(22) Filed: Jun. 8, 2016

Related U.S. Application Data
(63) Continuation of application No. PCT/JP2014/082418, filed on Dec. 8, 2014.

(30) Foreign Application Priority Data
Dec. 9, 2013 (JP) 2013-254423
Apr. 15, 2014 (JP) 2014-083667
Jun. 17, 2014 (JP) 2014-124036

(51) Int. Cl. C08G 63/91; H01L 31/049; C07C 317/40; C08J 5/18; C07C 257/08

(52) U.S. Cl. CPC C08G 63/916; C08J 5/18; C07C 257/08; C07C 317/40; H01L 31/049; C08J 2367/02

(57) ABSTRACT

A polyester resin composition is provided with which irritant gases do not occur in production and no increase in the viscosity of a polyester resin is exhibited even when a terminal blocking agent is contained. The composition includes an iminoether compound of Formula (1) and a polyester. Further provided are an iminoether compound, a method for producing a carboxylic acid ester, a polyester film, a back sheet for a solar cell module, and a solar cell module. R² represents an optionally substituted alkyl, cycloalkyl, aryl or alkoxy group; R³ represents a specific alkyl or aryl group; and R¹¹, R¹² and R¹³ each independently represents a hydrogen atom or an optionally substituted alkyl or aryl group.

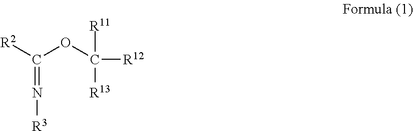

### CROSS-REFERENCE TO RELATED APPLICATIONS

This application is a Continuation of PCT International Application No. PCT/JP2014/082418, filed on Dec. 8, 2014, which claims priority under 35 U.S.C. Section 119(a) to Japanese Patent Application No. 2013-254423 filed on Dec. 9, 2013, Japanese Patent Application No. 2014-083667 filed on Apr. 15, 2014, and Japanese Patent Application No. 2014-124036 filed on Jun. 17, 2014. Each of the above applications is hereby expressly incorporated by reference, in its entirety, into the present application.

### BACKGROUND OF THE INVENTION

1. Field of the Invention

The present invention relates to a polyester resin composition containing an iminoether compound, a back sheet for a solar cell module having a polyester film prepared from the polyester resin composition, and a solar cell module having the back sheet for a solar cell module laminated thereon. Further, the present invention relates to a novel iminoether compound and a method for producing a carboxylic acid ester.

2. Description of the Related Art

A solar cell module generally has a structure in which glass or a front sheet/a transparent filling material (sealing material)/a solar cell element/a sealing material/a back sheet are laminated in this order on the light-receiving surface side onto which sunlight is incident. Specifically, the solar cell element is generally configured to have a structure in which the solar cell element is embedded in a resin (sealing material) such as EVA (an ethylene-vinyl acetate copolymer) and the like, and a protective sheet for a solar cell is further adhered thereon. A protective sheet for a solar cell, in particular, a back sheet for a solar cell module which becomes the outermost layer is understood to be in an environment of exposure to outdoors weather such as rain, wind or direct sunlight for long periods of time, and therefore is required to have excellent weather resistance (wet heat resistance and heat resistance).

Conventionally, a polyester film, in particular, a polyethylene terephthalate (which is hereinafter also referred to as PET) film has been used in the back sheet for a solar cell module. A polyester film has excellent heat resistance, mechanical characteristics, chemical resistance, and the like and therefore is widely and industrially used. However, these films have poor hydrolysis resistance and consequently undergo lowering of the molecular weight thereof due to hydrolysis, and exhibit the progress of embrittlement resulting in deterioration of mechanical properties or the like. As a consequence, polyester films have not been able to retain a practical strength over a long period of time as a back sheet for a solar cell.

As a method for solving this problem, there is known a method of blocking the carboxylic acid remaining at the terminal of the polyester. As a terminal blocking agent for blocking the carboxylic acid remaining at the terminal of the polyester, there may be mentioned, for example, a carbodiimide compound or a cyclic iminoether compound. For example, JP2010-31174A discloses a polyester film which contains a carbodiimide compound or a cyclic iminoether compound. In this literature, it has been proposed to improve the dimensional stability or hydrolysis resistance of the polyester by reacting a carbodiimide compound or a cyclic iminoether compound with the carboxylic acid of the polyester terminal. In addition, JP2010-31174A has employed an oxazoline compound or an oxazine compound as the cyclic iminoether compound.

However, in the case of using the carbodiimide compound described in JP2010-31174A, there is a problem associated with volatilization of free isocyanates. Such volatile gases are an irritant gas and lead to deterioration of the polyester film manufacturing environment, which in turn presents a problem.

For these reasons, using a cyclic iminoether compound as a terminal blocking agent has been considered. However, cyclic iminoether compounds have been known to undergo self-polymerization, and it has been clearly revealed by the investigation of the present inventors that the self-polymerization product of a cyclic iminoether compound is present as a gel component in a polyester resin. Such a gel component contributes to an increase in the viscosity of the polyester resin and therefore leads to deterioration of the surface state of the polyester film, which consequently becomes a problem.

In order to solve these problems in the conventional art, the present inventors have conducted research with the aim of providing a polyester resin composition with which irritant gases do not occur in the production process of a polyester film containing a terminal blocking agent and with which no increase in the viscosity of a polyester resin is exhibited even in the case of a terminal blocking agent being contained.

### SUMMARY OF THE INVENTION

As a result of extensive studies to solve the above-mentioned problems, the present inventors have found that when an iminoether compound having a certain structure is used as a terminal blocking agent, an occurrence of irritant gases can be inhibited in the polyester film production process and a polyester film having favorable film surface state can also be obtained.

Specifically, the present invention has the following configuration.

[1] A polyester resin composition including an iminoether compound represented by the following General Formula (1) and a polyester.

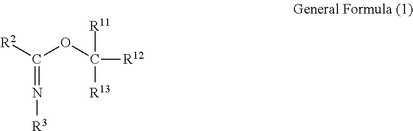

In General Formula (1), R² represents an alkyl group which may have a substituent, a cycloalkyl group which may have a substituent, an aryl group which may have a substituent or an alkoxy group which may have a substituent, R³ represents an alkyl group represented by the following General Formula (2) or an aryl group represented by the following General Formula (3), and R¹¹, R¹² and R¹³ each independently represent a hydrogen atom, an alkyl group which may have a substituent or an aryl group which may have a substituent; or alternatively, R², R³, R¹¹, R¹² and R¹³ may combine together to form a ring; provided that, in the case where R³ is represented by the following General Formula (2), a bond which is formed between at least one of R¹¹, R¹², or R¹³ and at least one of R³¹, R³², or R³³ is a bond in which the number of connecting atoms is 2 or more.

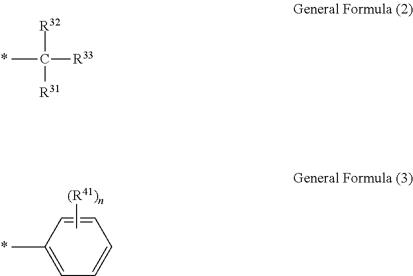

In General Formula (2), R³¹, R³² and R³³ each independently represent a hydrogen atom or a substituent; R³¹, R³² and R³³ may combine together to form a ring; in General Formula (3), R⁴¹ represents a substituent, and in the case where a plurality of R⁴¹'s are present, they may be the same or different; n represents an integer of 0 to 5; and * in General Formulae (2) and (3) represents a site which is bonded to a nitrogen atom.

[2] The polyester resin composition described in [1], wherein the iminoether compound is represented by the following General Formula (4);

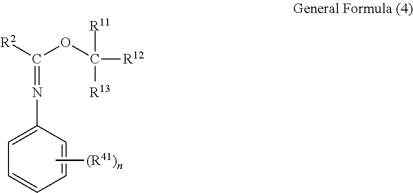

In General Formula (4), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; and n represents an integer of 0 to 5.

[3] The polyester resin composition described in [1] or [2], wherein the iminoether compound is represented by the following General Formula (5);

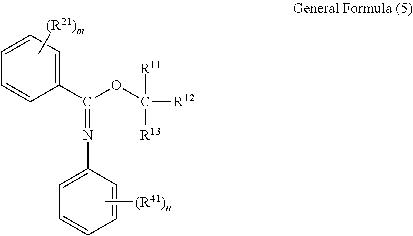

In General Formula (5), R¹¹, R¹² and R¹³ each independently represent a hydrogen atom, an alkyl group which may have a substituent or an aryl group which may have a substituent; R²¹ and R⁴¹ each independently represent a substituent; and in the case where a plurality of either of R²¹'s and R⁴¹'s are present, they may be the same or different; n represents an integer of 0 to 5, and m represents an integer of 0 to 5.

[4] The polyester resin composition described in any one of [1] to [3], wherein the iminoether compound is represented by the following General Formula (6);

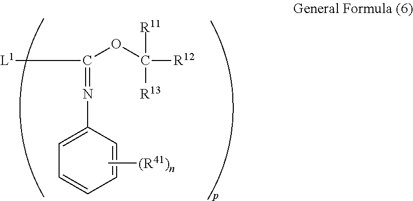

In General Formula (6), R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; n represents an integer of 0 to 5; p represents an integer of 2 to 4, and L¹ represents a p-valent group in which the end bonded to the carbon atom is an alkylene portion which may have a substituent, a cycloalkylene portion which may have a substituent, an arylene portion which may have a substituent, or an alkoxylene portion which may have a substituent.

[5] The polyester resin composition described in any one of [1] to [4], wherein the iminoether compound is represented by the following General Formula (7);

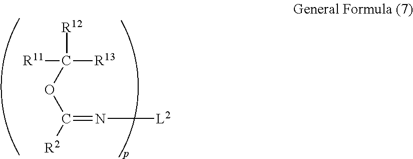

In General Formula (7), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; p represents an integer of 2 to 4, and L² represents a p-valent group in which the end bonded to the nitrogen atom is an arylene portion which may have a substituent, or a cycloalkylene portion which may have a substituent.

[6] The polyester resin composition described in any one of [1] to [5], wherein the iminoether compound is represented by the following General Formula (8);

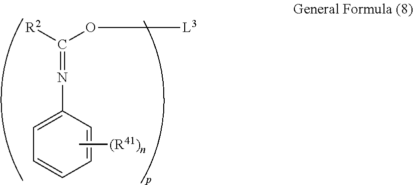

In General Formula (8), R² represents an alkyl group which may have a substituent, a cycloalkyl group which may have a substituent, an aryl group which may have a substituent or an alkoxy group which may have a substituent, R⁴¹ represents a substituent, and in the case where a plurality of R⁴¹'s are present, they may be the same or different; n represents an integer of 0 to 5; p represents an integer of 2 to 4, and L³ represents a p-valent group in which the end bonded to the oxygen atom is an alkylene portion; provided that some or all of hydrogen atoms in the alkylene portion of L³ may be substituted with an alkyl group which may have a substituent or an aryl group which may have a substituent.

[7] The polyester resin composition described in any one of [1] to [6], wherein the iminoether compound is prepared using an orthoester compound.
[8] The polyester resin composition described in any one of [1] to [7], wherein the composition includes 0.05 to 5% by mass of the iminoether compound with respect to the polyester.
[9] The polyester resin composition described in any one of [1] to [8], wherein a component derived from a carboxylic acid of the polyester is a component derived from an aromatic dibasic acid or an ester-forming derivative thereof.
[10] An iminoether compound represented by the following General Formula (4);

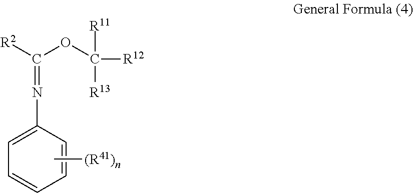

In General Formula (4), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; and n represents an integer of 0 to 5.

[11] The iminoether compound described in [10], wherein the compound is represented by the following General Formula (5);

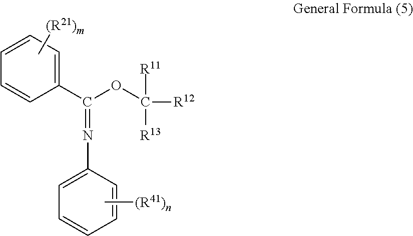

In General Formula (5), R¹¹, R¹² and R¹³ each independently represent a hydrogen atom, an alkyl group which may have a substituent or an aryl group which may have a substituent; R²¹ and R⁴¹ each independently represent a substituent; and in the case where a plurality of either of R²¹'s and R⁴¹'s are present, they may be the same or different; n represents an integer of 0 to 5, and m represents an integer of 0 to 5.

[12] The iminoether compound described in [10] or [11], wherein the compound is represented by the following General Formula (6);

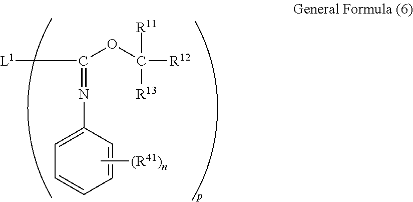

In General Formula (6), R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; n represents an integer of 0 to 5; p represents an integer of 2 to 4, and L¹ represents a p-valent group in which the end bonded to the carbon atom is an alkylene portion which may have a substituent, a cycloalkylene portion which may have a substituent, an arylene portion which may have a substituent, or an alkoxylene portion which may have a substituent.

[13] The iminoether compound described in any one of [10] to [12], wherein the compound is represented by the following General Formula (7);

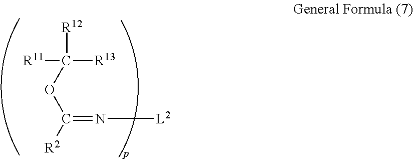

In General Formula (7), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; p represents an integer of 2 to 4, and L² represents a p-valent group in which the end bonded to the nitrogen atom is an arylene portion which may have a substituent.

[14] The iminoether compound described in any one of [10] to [13], wherein the compound is represented by the following General Formula (8);

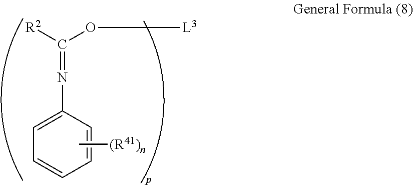

In General Formula (8), R² represents an alkyl group which may have a substituent, a cycloalkyl group which may have a substituent, an aryl group which may have a substituent or an alkoxy group which may have a substituent, R⁴¹ represents a substituent, and in the case where a plurality of R⁴¹'s are present, they may be the same or different; n represents an integer of 0 to 5; p represents an integer of 2 to 4, and L³ represents a p-valent group in which the end bonded to the oxygen atom is an alkylene portion; provided that some or all of hydrogen atoms in the alkylene portion of L³ may be substituted with an alkyl group which may have a substituent or an aryl group which may have a substituent.

[15] The iminoether compound described in any one of [10] to [14], wherein the compound is prepared using an orthoester compound.
[16] A method for producing a carboxylic acid ester, including reacting the iminoether compound described in any one of [10] to [15] with a compound having a carboxylic acid group at a temperature of 100 to 350° C. to produce a carboxylic acid ester.
[17] A polyester film fabricated from the polyester resin composition described in any one of [1] to [9].
[18] The polyester film described in [17], wherein the film is biaxially stretched.
[19] A back sheet for a solar cell module using the polyester film described in [17] or [18].
[20] A solar cell module using the back sheet for a solar cell module described in [19].

According to the present invention, it is possible to suppress the occurrence of irritant gases in the production process of a polyester film containing a terminal blocking agent. Thus, it is possible to enhance the work safety during the polyester film production. Moreover, according to the present invention, even in the case where the terminal blocking agent is contained, it is possible to obtain a polyester film having a satisfactory film surface state without increasing the viscosity of the polyester resin.

### DESCRIPTION OF THE PREFERRED EMBODIMENTS

Hereinafter, the present invention will be described in detail. The description of the constitutive elements as described below is based on representative embodiments or specific examples of the present invention, but the present invention should not be limited thereto. Further, the numerical range expressed by the wording “(a lower limit) to (an upper limit)” means a range that falls between the former number indicating the lower limit of the range and the latter number indicating the upper limit thereof.

(Iminoether Compound)

The iminoether compound used in the present invention is represented by the following General Formula (1).

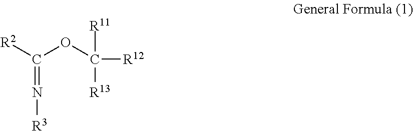

In General Formula (1), R² represents an alkyl group which may have a substituent, a cycloalkyl group which may have a substituent, an aryl group which may have a substituent or an alkoxy group which may have a substituent, R³ represents an alkyl group represented by the following General Formula (2), or an aryl group represented by the following General Formula (3), and R¹¹, R¹² and R¹³ each independently represent a hydrogen atom, an alkyl group which may have a substituent or an aryl group which may have a substituent. Alternatively, R², R³, R¹¹, R¹² and R¹³ may combine together to form a ring. However, in the case where R³ is represented by the following General Formula (2), a bond which is formed between at least one of R¹¹, R¹², or R¹³ and at least one of R³¹, R³², or R³³ is a bond in which the number of connecting atoms is 2 or more.

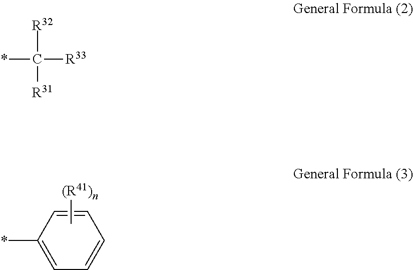

In General Formula (2), R³¹, R³² and R³³ each independently represent a hydrogen atom or a substituent. R³¹, R³² and R³³ may combine together to form a ring. In General Formula (3), R⁴¹ represents a substituent, and in the case where a plurality of R⁴¹'s are present, they may be the same or different, n represents an integer of 0 to 5. Furthermore, * in General Formulae (2) and (3) represents a site which is bonded to a nitrogen atom.

In General Formula (1), the alkyl group represented by R² is preferably an alkyl group having 1 to 20 carbon atoms, and more preferably an alkyl group having 1 to 12 carbon atoms. The alkyl group represented by R² may be linear or branched. Moreover, the alkyl group represented by R² may be a cycloalkyl group. Examples of the alkyl group represented by R² may include a methyl group, an ethyl group, an n-propyl group, an iso-propyl group, an n-butyl group, a tert-butyl group, a sec-butyl group, an iso-butyl group, an n-pentyl group, a sec-pentyl group, an iso-pentyl group, an n-hexyl group, a sec-hexyl group, an iso-hexyl group, and a cyclohexyl group. Of these, a methyl group, an ethyl group, an n-propyl group, an iso-propyl group, an iso-butyl group, and a cyclohexyl group are more preferred.

The alkyl group represented by R² may further have a substituent. Examples of the substituent may include the above-mentioned alkyl group, an aryl group, an alkoxy group, a halogen atom, a nitro group, an amide group, a hydroxyl group, an ester group, an ether group, and an aldehyde group. Furthermore, the number of carbon atoms of the alkyl group represented by R² indicates the number of carbon atoms that does not include a substituent.

The aryl group represented by R² is preferably an aryl group having 6 to 20 carbon atoms, and more preferably an aryl group having 6 to 12 carbon atoms. Examples of the aryl group represented by R² may include a phenyl group and a naphthyl group. Of these, a phenyl group is particularly preferred. The aryl group represented by R² may further have a substituent. Furthermore, as the substituent there may be exemplified those substituents described above, but the substituent is not particularly limited as long as it may allow to proceed with the reaction of an iminoether group with a carboxyl group. Moreover, the number of carbon atoms of the aryl group represented by R² indicates the number of carbon atoms that does not include a substituent.

The alkoxy group represented by R² is preferably an alkoxy group having 1 to 20 carbon atoms, more preferably an alkoxy group having 1 to 12 carbon atoms, and particularly preferably an alkoxy group having 2 to 6 carbon atoms. The alkoxy group represented by R² may be linear or branched or may be cyclic. Preferred examples of the alkoxy group represented by R² may include groups in which —O— is linked to the end of the alkyl group represented by R². The alkoxy group represented by R² may further have a substituent. Furthermore, as the substituent there may be exemplified those substituents described above, but the substituent is not particularly limited as long as it may allow to proceed with the reaction of an iminoether group with a carboxyl group. Further, the number of carbon atoms of the alkoxy group represented by R² indicates the number of carbon atoms that does not include a substituent.

R³ represents an alkyl group represented by the above-mentioned General Formula (2), or an aryl group represented by the above-mentioned General Formula (3). In General Formula (2), R³¹, R³² and R³³ each independently represent a hydrogen atom or a substituent. In the case where R³¹, R³² and R³³ are a substituent, they may combine together to form a ring. Examples of the substituent may include the above-mentioned alkyl group, an aryl group, an alkoxy group, a halogen atom, a nitro group, an amide group, a hydroxyl group, an ester group, an ether group, and an aldehyde group. All of R³¹, R³² and R³³ may be a hydrogen atom or may be the same or different substituents. Moreover, the alkyl group represented by General Formula (2) may be linear or branched. Furthermore, the alkyl group represented by General Formula (2) may be a cycloalkyl group.

In General Formula (3), R⁴¹ represents a substituent, and n represents an integer of 0 to 5. In the case where n is 2 or more, R⁴¹ may be the same or different. As the substituent, there may be exemplified those substituents described above. Furthermore, n is more preferably 0 to 3, and even more preferably 0 to 2.

R¹¹, R¹² and R¹³ each independently represent a hydrogen atom, an alkyl group which may have a substituent or an aryl group which may have a substituent. As the alkyl and aryl groups, there may be exemplified those alkyl and aryl groups which R² can take.

R², R³, R¹¹, R¹² and R¹³ preferably do not combine to form a ring, but R², R³, R¹¹, R¹² and R¹³ may combine together to form a ring. For example, in the case where R³ is represented by the above-mentioned General Formula (3), R⁴¹ and at least one of R¹¹, R¹², or R¹³ may preferably combine together to form a ring, and the benzene ring and the ring containing any one of R¹¹ to R¹³ may form a fused ring. In the case where R³ is represented by the above-mentioned General Formula (3), it is preferred that R⁴¹ and at least one of R¹¹, R¹², or R¹³ do not combine to form a ring.

However, in the case where R³ is represented by the above-mentioned General Formula (2), a bond which is formed between at least one of R¹¹, R¹², or R¹³ and at least one of R³¹, R³², or R³³ is a bond in which the number of connecting atoms is 2 or more. In the case where R³ is represented by the above-mentioned General Formula (2), a bond which is formed between one of R¹¹ to R¹³ and one of R³¹ to R³³ is a bond in which the number of connecting atoms is 2 or more and is additionally preferably a double bond. In the case where R³ is represented by the above-mentioned General Formula (2), at least one of R¹¹, R¹², or R¹³ and at least one of R³¹, R³², or R³³ preferably do not combine to form a ring.

General Formula (1) may include a repeating unit. In this case, R², R³ or at least one of R¹¹, R¹², or R¹³ is a repeating unit. It is preferred that an iminoether portion is included in this repeating unit.

Moreover, the iminoether compound used in the present invention is preferably represented by the following General Formula (4).

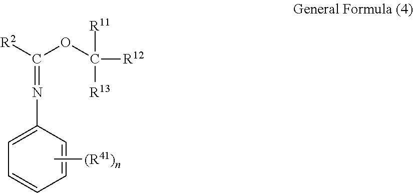

In General Formula (4), R² represents an alkyl group which may have a substituent, a cycloalkyl group which may have a substituent, an aryl group which may have a substituent or an alkoxy group which may have a substituent, and R¹¹, R¹² and R¹³ each independently represent a hydrogen atom, an alkyl group which may have a substituent or an aryl group which may have a substituent. R⁴¹ represents a substituent, and in the case where a plurality of R⁴¹'s are present, they may be the same or different. n represents an integer of 0 to 5.

R², R¹¹, R¹² and R¹³ in General Formula (4) each have the same meaning as R², R¹¹, R¹² and R¹³ in General Formula (1), and preferred ranges thereof are also the same. Moreover, R⁴¹ in General Formula (4) has the same meaning as R⁴¹ in General Formula (3), and preferred ranges thereof are also the same. n is preferably 0 to 3, and more preferably 0 to 2.

The iminoether compound used in the present invention is preferably represented by the following General Formula (5).

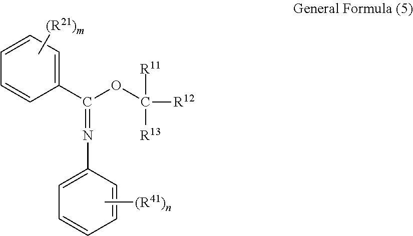

In General Formula (5), R¹¹, R¹² and R¹³ each independently represent a hydrogen atom, an alkyl group which may have a substituent or an aryl group which may have a substituent. R²¹ and R⁴¹ each independently represent a substituent. In the case where a plurality of either of R²¹'s and R⁴¹'s are present, they may be the same or different. n represents an integer of 0 to 5, and m represents an integer of 0 to 5.

R¹¹, R¹² and R¹³ in General Formula (5) each have the same meaning as R¹¹, R¹² and R¹³ in General Formula (1), and preferred ranges thereof are also the same. R⁴¹ in General Formula (5) has the same meaning as R⁴¹ in General Formula (3), and preferred ranges thereof are also the same. Furthermore, the same substituent for R⁴¹ in General Formula (3) may be exemplified for R²¹.

Moreover, in General Formula (5), n is preferably 0 to 3, and more preferably 0 to 2. Furthermore, m is preferably 0 to 3, and more preferably 0 to 2.

The iminoether compound used in the present invention is preferably represented by the following General Formula (6).

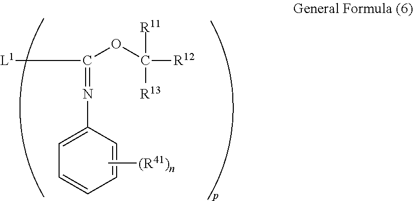

In General Formula (6), R¹¹, R¹² and R¹³ each independently represent a hydrogen atom, an alkyl group which may have a substituent or an aryl group which may have a substituent. R⁴¹ represents a substituent, and in the case where a plurality of R⁴¹'s are present, they may be the same or different. n represents an integer of 0 to 5. p represents an integer of 2 to 4, and L¹ represents a p-valent group in which the end bonded to the carbon atom is an alkylene portion which may have a substituent, a cycloalkylene portion which may have a substituent, an arylene portion which may have a substituent, or an alkoxylene portion which may have a substituent.

R¹¹, R¹² and R¹³ in General Formula (6) each have the same meaning as R¹¹, R¹² and R¹³ in General Formula (1), and preferred ranges thereof are also the same. Moreover, R⁴¹ in General Formula (6) has the same meaning as R⁴¹ in General Formula (3), and preferred ranges thereof are also the same. Moreover, n is preferably 0 to 3, and more preferably 0 to 2.

In General Formula (6), L¹ represents a p-valent group in which the end bonded to the carbon atom is an alkylene portion which may have a substituent, a cycloalkylene portion which may have a substituent, an arylene portion which may have a substituent, or an alkoxylene portion which may have a substituent. p represents an integer of 2 to 4. p is preferably 2 or 3.

Specific examples of the divalent group may include, for example, an alkylene group which may have a substituent, a cycloalkylene group which may have a substituent, an arylene group which may have a substituent, and an alkoxylene group which may have a substituent. Moreover, examples thereof may include groups in which the end bonded to the carbon atom is an alkylene portion which may have a substituent, a cycloalkylene portion which may have a substituent, an arylene portion which may have a substituent, or an alkoxylene portion which may have a substituent, and include, as a partial structure, at least one selected from —SO₂—, —CO—, a substituted or unsubstituted alkylene portion, a substituted or unsubstituted alkenylene portion, an alkynylene portion, a substituted or unsubstituted phenylene portion, a substituted or unsubstituted biphenylene portion, a substituted or unsubstituted naphthylene portion, —O—, —S— or —SO—.

Preferably, for example, examples thereof may include substituted or unsubstituted phenylene, substituted or unsubstituted biphenylene, substituted or unsubstituted naphthylene, ethylene, n-butylene, substituted or unsubstituted cyclohexylene, substituted or unsubstituted —C₆H₁₀—C₆H₁₀—, substituted or unsubstituted —C₆H₁₀—CH₂—C₆H₁₀—, substituted or unsubstituted —C₆H₅—C(CH₃)₂—C₆H₅—, substituted or unsubstituted —C₆H₅—CH₂—C₆H₅—, substituted or unsubstituted —C₆H₅—C(O)—C₆H₅—, substituted or unsubstituted —C₆H₅—O—C₆H₅—, substituted or unsubstituted —C₆H₅—S—C₆H₅—, substituted or unsubstituted —C₆H₅—SO₂—C₆H₅—, substituted or unsubstituted —C₆H₅—C(CF₃)₂—C₆H₅—, substituted or unsubstituted —C₆H₅—NHC(O)—C₆H₅—, substituted or unsubstituted —C₆H₅—O—C₆H₅—C(CH₃)₂—C₆H₅—O—C₆H₅—, substituted or unsubstituted —C₆H₅—O—C₆H₅—C(O)—C₆H₅—O—C₆H₅—, substituted or unsubstituted —C₆H₅—O—C₆H₅—SO₂—C₆H₅—O—C₆H₅—, substituted or unsubstituted —C₆H₅—O—C₆H₅—S—C₆H₅—O—C₆H₅—, substituted or unsubstituted —C₆H₅—O—(C₆H₅)₂—O—C₆H₅—, substituted or unsubstituted —C₆H₅—O—C₆H₅—C(CF₃)₂—C₆H₅—O—C₆H₅—, and the like.

Specific examples of the trivalent group may include, for example, groups obtained by the removal of one hydrogen atom from the group having a substituent among the groups mentioned as examples of the divalent group.

Specific examples of the tetravalent group may include, for example, groups obtained by the removal of two hydrogen atoms from the group having a substituent among the groups mentioned as examples of the divalent group.

In the present invention, by setting p to fall within the range of 2 to 4, it is possible to make a compound having two or more iminoether portions in one molecule and therefore to exhibit superior terminal blocking effect. Furthermore, by making the compound having two or more iminoether portions in one molecule, it is possible to lower an iminoether value (total molecular weight/the number of functional groups of iminoether) and therefore it is possible to efficiently react the iminoether compound with the terminal carboxyl group of the polyester.

The iminoether compound used in the present invention is preferably represented by the following General Formula (7).

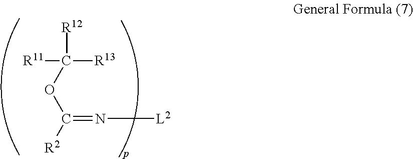

In General Formula (7), R² represents an alkyl group which may have a substituent, a cycloalkyl group which may have a substituent, an aryl group which may have a substituent or an alkoxy group which may have a substituent, and R¹¹, R¹² and R¹³ each independently represent a hydrogen atom, an alkyl group which may have a substituent or an aryl group which may have a substituent. Moreover, p represents an integer of 2 to 4, and L² represents a p-valent group in which the end bonded to the nitrogen atom is an arylene portion which may have a substituent, or a cycloalkylene portion which may have a substituent.

R², R¹¹, R¹² and R¹³ in General Formula (7) each have the same meaning as R², R¹¹, R¹² and R¹³ in General Formula (1), and preferred ranges thereof are also the same.

In General Formula (7), L² represents a p-valent group in which the end bonded to the nitrogen atom is an arylene portion which may have a substituent, or a cycloalkylene portion which may have a substituent. L² is preferably a p-valent group in which the end bonded to the nitrogen atom is an arylene portion which may have a substituent. p represents an integer of 2 to 4. p is preferably 2 or 3.

Specific examples of L² may include an arylene group which may have a substituent and a cycloalkylene group which may have a substituent. Moreover, examples thereof may include groups in which the end bonded to the nitrogen atom is an arylene portion which may have a substituent or a cycloalkylene portion which may have a substituent, and include, as a partial structure, at least one selected from —SO₂—, —CO—, a substituted or unsubstituted alkylene portion, a substituted or unsubstituted alkenylene portion, an alkynylene portion, a substituted or unsubstituted phenylene portion, a substituted or unsubstituted biphenylene portion, a substituted or unsubstituted naphthylene portion, —O—, —S— or —SO—.

Preferably, for example, examples thereof may include substituted or unsubstituted phenylene, substituted or unsubstituted biphenylene, substituted or unsubstituted naphthylene, substituted or unsubstituted cyclohexylene, substituted or unsubstituted —C₆H₁₀—C₆H₁₀—, substituted or unsubstituted —C₆H₁₀—CH₂—C₆H₁₀—, substituted or unsubstituted —C₆H₅—C(CH₃)₂—C₆H₅—, substituted or unsubstituted —C₆H₅—CH₂—C₆H₅—, substituted or unsubstituted —C₆H₅—C(O)—C₆H₅—, substituted or unsubstituted —C₆H₅—O—C₆H₅—, substituted or unsubstituted —C₆H₅—S—C₆H₅—, substituted or unsubstituted —C₆H₅—SO₂—C₆H₅—, substituted or unsubstituted —C₆H₅—C(CF₃)₂—C₆H₅—, substituted or unsubstituted —C₆H₅—NHC(O)—C₆H₅—, substituted or unsubstituted —C₆H₅—O—C₆H₅—C(CH₃)₂—C₆H₅—OC₆H₅—, substituted or unsubstituted —C₆H₅—O—C₆H₅—C(O)—C₆H₅—O—C₆H₅—, substituted or unsubstituted —C₆H₅—O—C₆H₅—SO₂—C₆H₅—OC₆H₅—, substituted or unsubstituted —C₆H₅—O—C₆H₅—S—C₆H₅—O—C₆H₅—, substituted or unsubstituted —C₆H₅—O—(C₆H₅)₂—O—C₆H₅—, substituted or unsubstituted —C₆H₅—O—C₆H₅—C(CF₃)₂—C₆H₅—O—C₆H₅—, and the like.

The iminoether compound used in the present invention is preferably represented by the following General Formula (8).

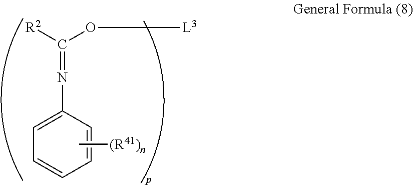

In General Formula (8), R² represents an alkyl group which may have a substituent, a cycloalkyl group which may have a substituent, an aryl group which may have a substituent or an alkoxy group which may have a substituent, and R⁴¹ represents a substituent, and in the case where a plurality of R⁴¹'s are present, they may be the same or different. n represents an integer of 0 to 5. Moreover, p represents an integer of 2 to 4, and L³ represents a p-valent group in which the end bonded to the oxygen atom is an alkylene portion. However, some or all of hydrogen atoms in the alkylene portion of L³ may be substituted with an alkyl group which may have a substituent or an aryl group which may have a substituent.

R² in General Formula (8) has the same meaning as R² in General Formula (1), and preferred ranges thereof are also the same. Moreover, R⁴¹ in General Formula (8) has the same meaning as R⁴¹ in General Formula (3), and preferred ranges thereof are also the same. Moreover, n is preferably 0 to 3, and more preferably 0 to 2.

In General Formula (8), L³ represents a p-valent group in which the end bonded to the oxygen atom is an alkylene portion. Some or all of hydrogen atoms in the alkylene portion of L³ may be substituted with an alkyl group which may have a substituent or an aryl group which may have a substituent. p represents an integer of 2 to 4. p is preferably 2 or 3.

Specific examples of L³ may include an alkylene group. Moreover, examples thereof may include groups in which the end bonded to the oxygen atom is an alkylene portion, and include, as a partial structure, at least one selected from —SO₂—, —CO—, a substituted or unsubstituted alkylene group, a substituted or unsubstituted alkenylene group, an alkynylene group, a substituted or unsubstituted phenylene group, a substituted or unsubstituted biphenylene group, a substituted or unsubstituted naphthylene group, —O—, —S— or —SO—.

Preferably, for example, examples thereof may include ethylene, n-butylene, substituted or unsubstituted —CH₂—C(CH₃)₂—CH₂—, substituted or unsubstituted —CH₂—C₆H₅—CH₂—, and the like.

The molecular weight per an iminoether portion of the iminoether compound is preferably 1000 or less, more preferably 750 or less, and even more preferably 500 or less. By taking the molecular weight per iminoether portion to fall within this range, it is possible to block the terminal carboxylic acid groups of the polyester at a low addition amount of the iminoether compound.

Preferred specific examples of General Formula (1) are shown below, but the present invention is not limited thereto.

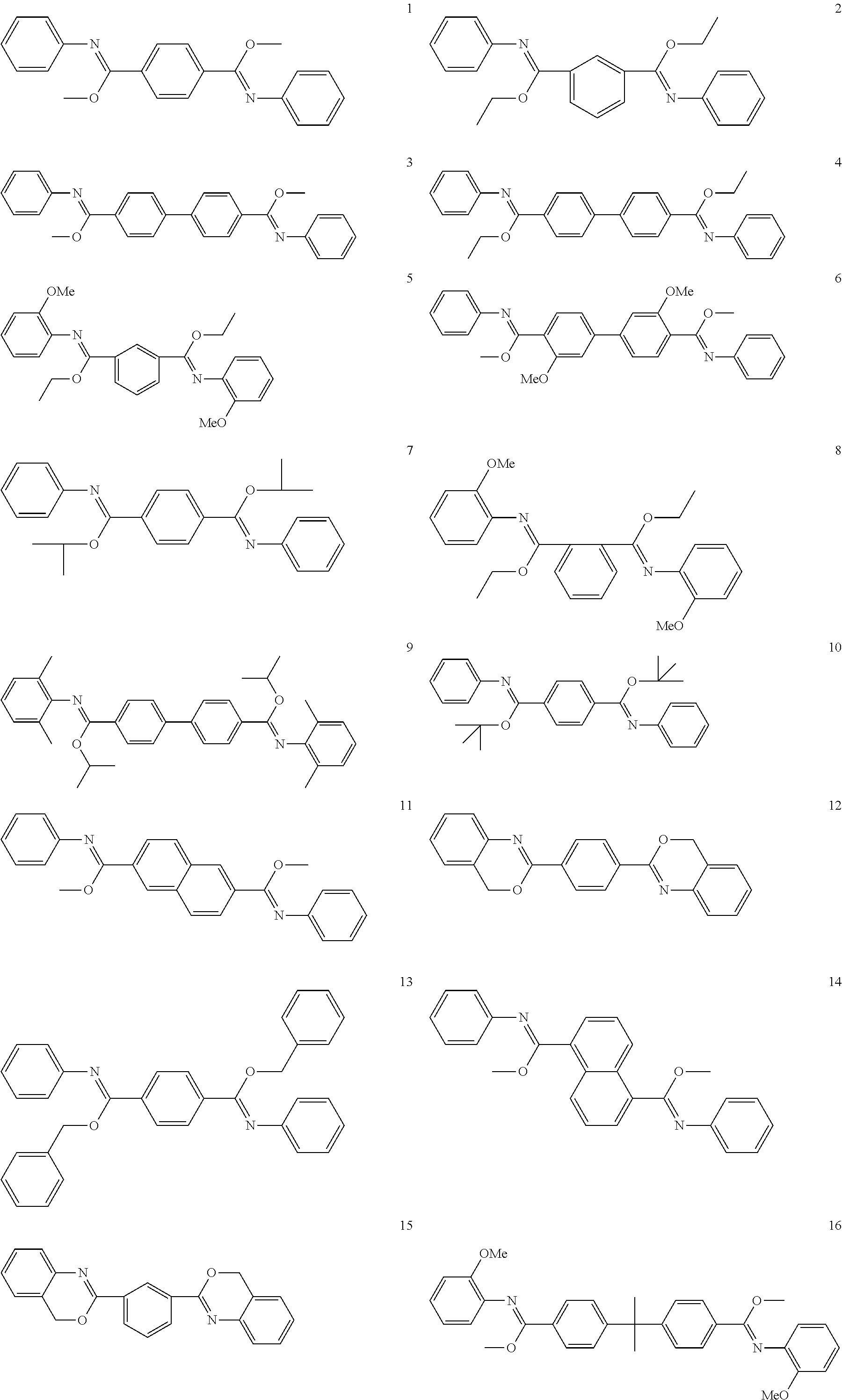

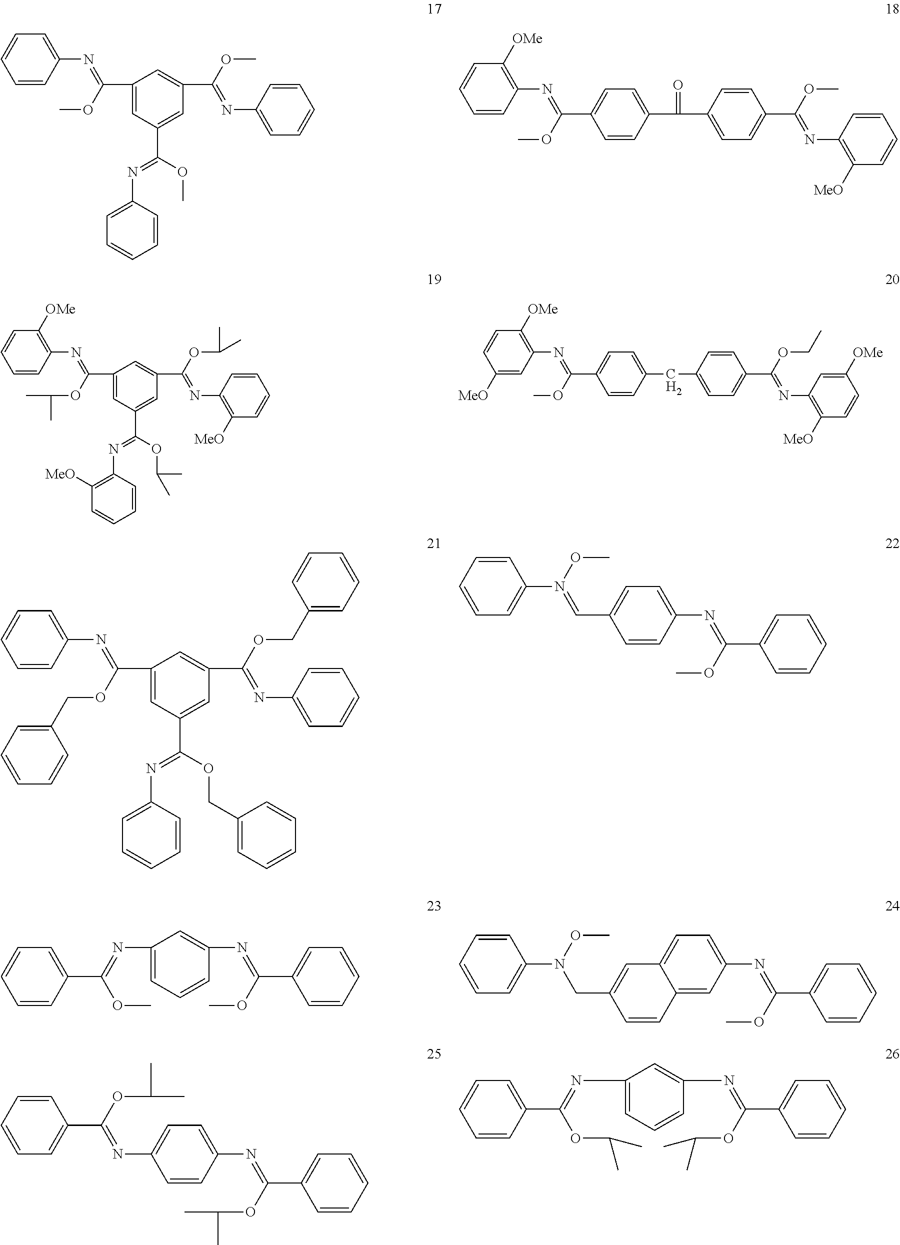

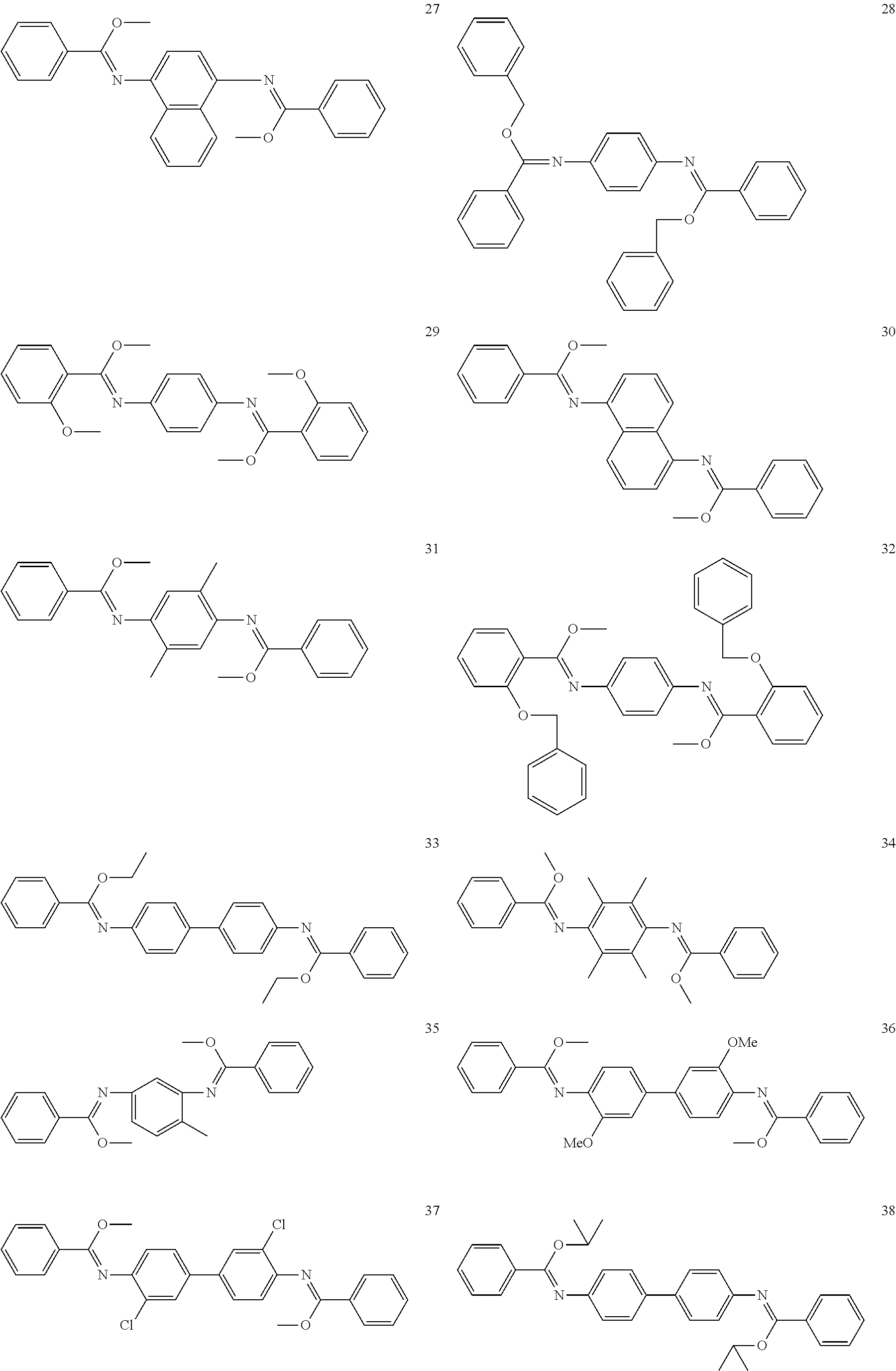

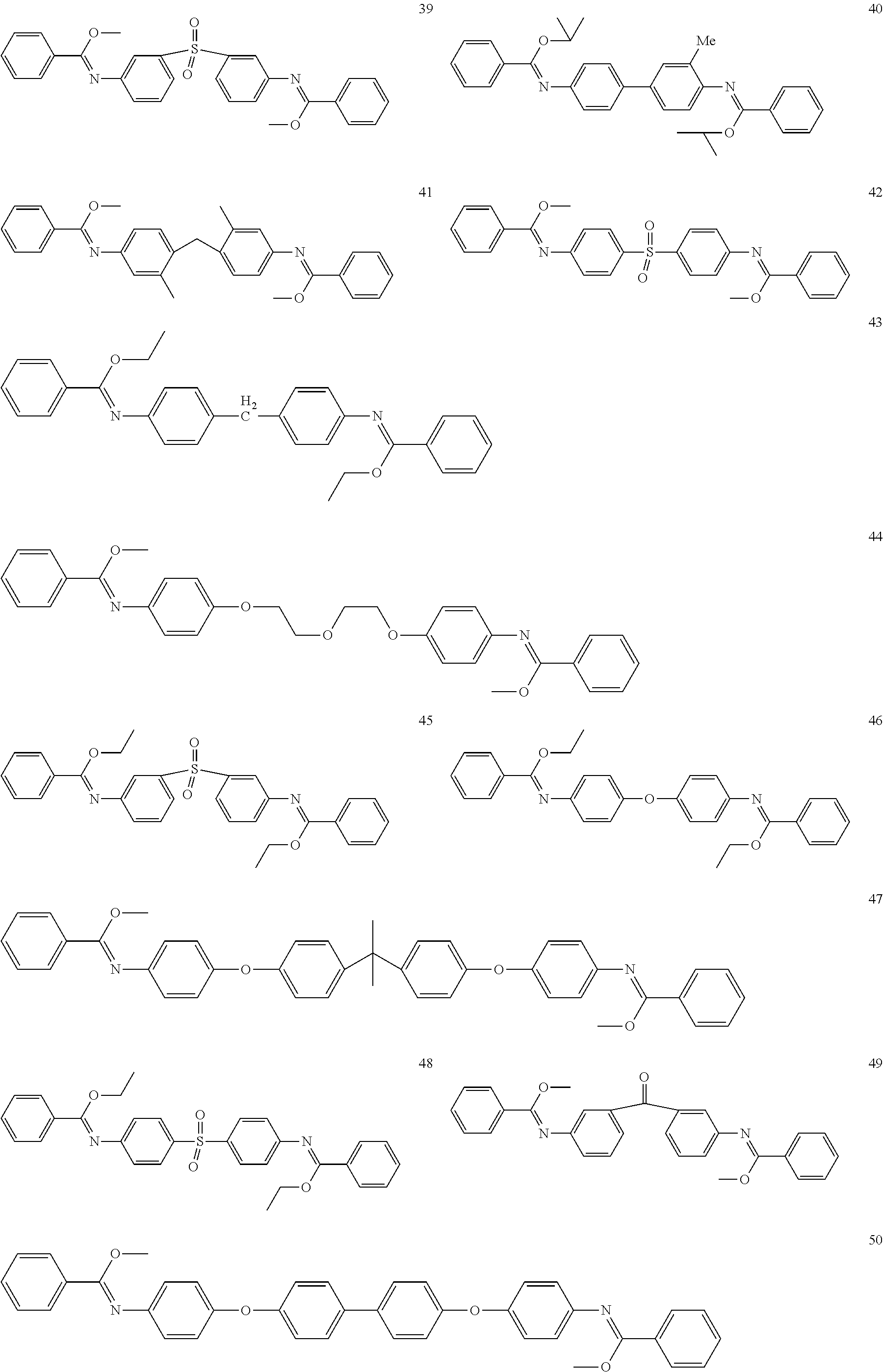

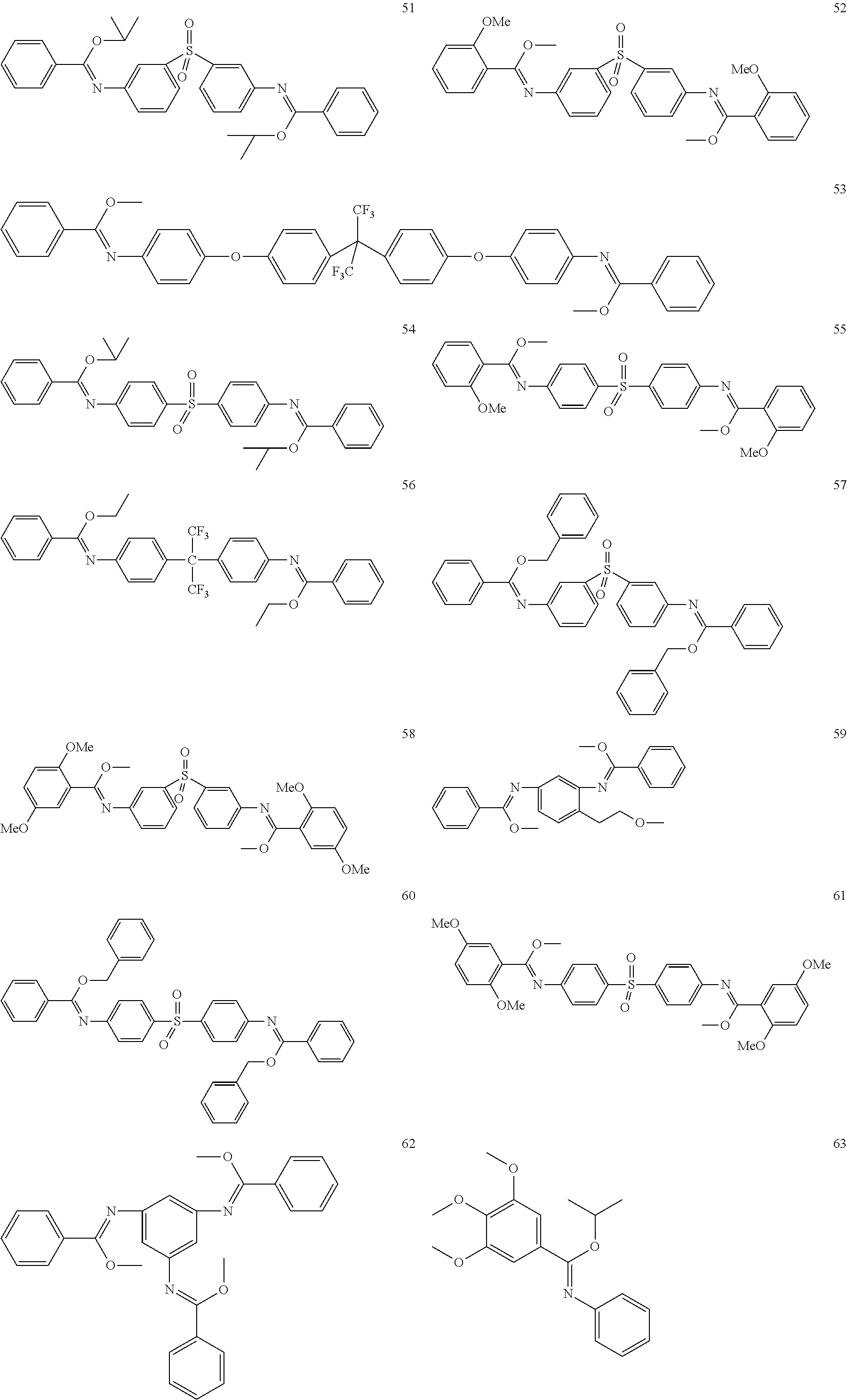

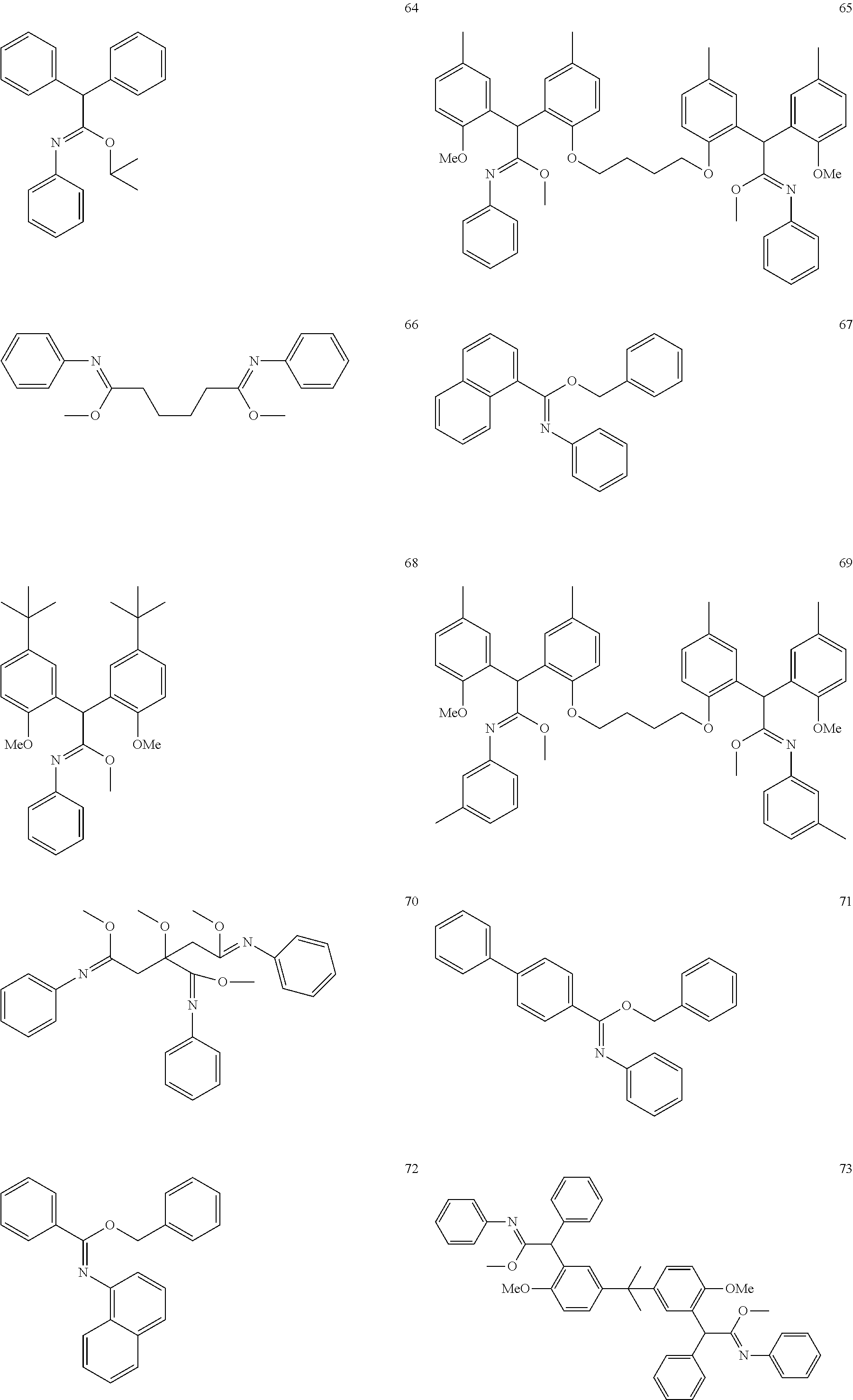

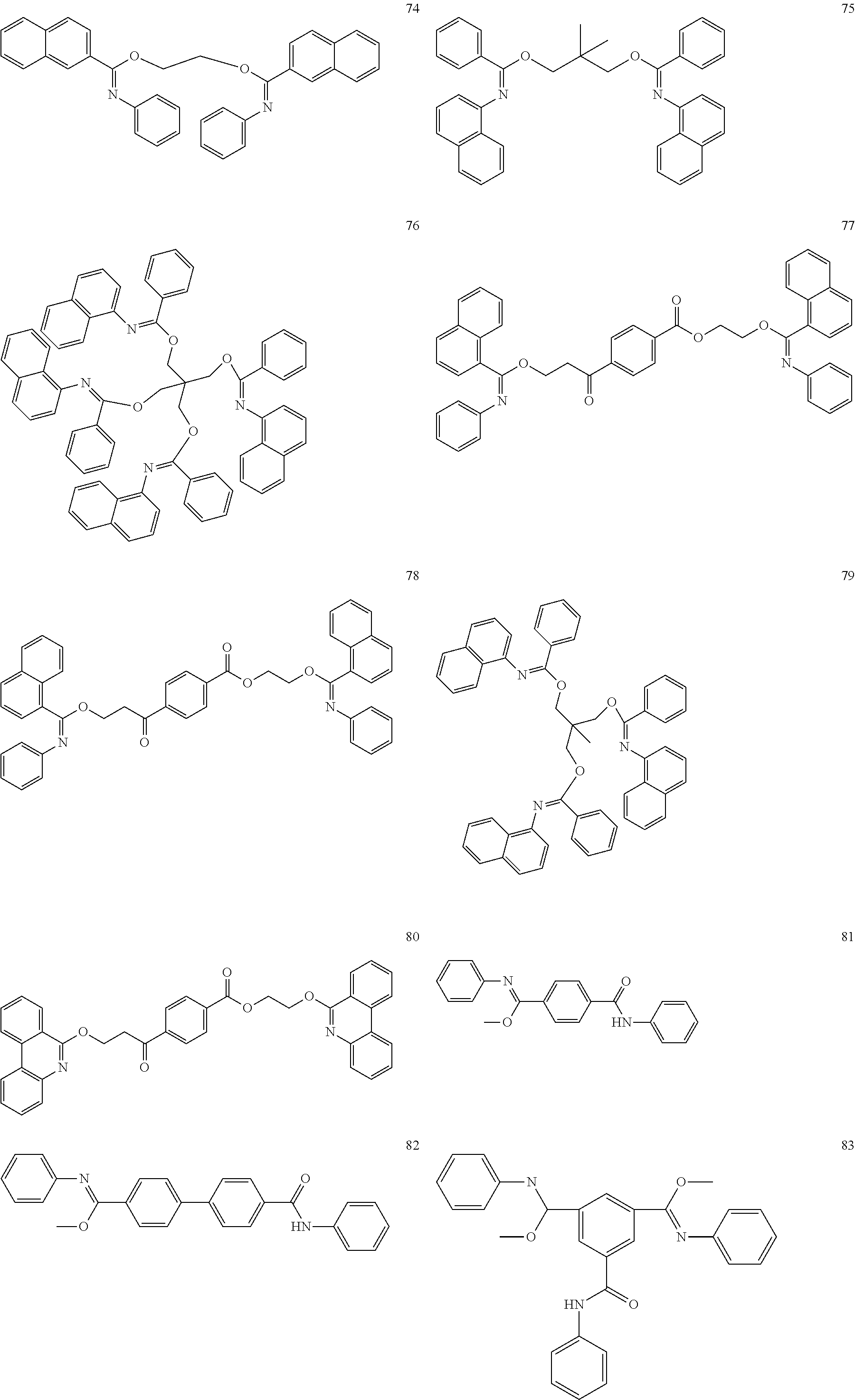

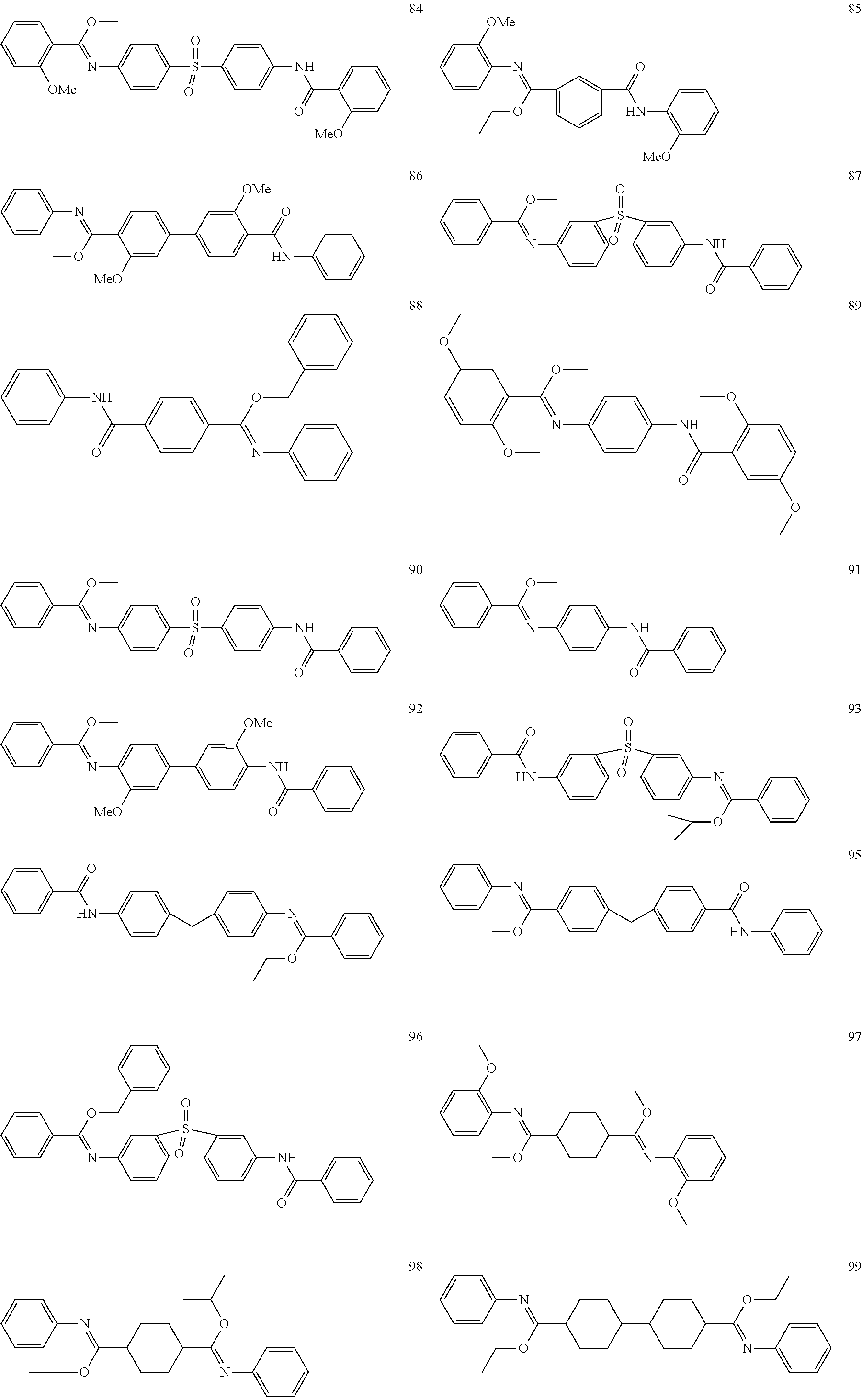

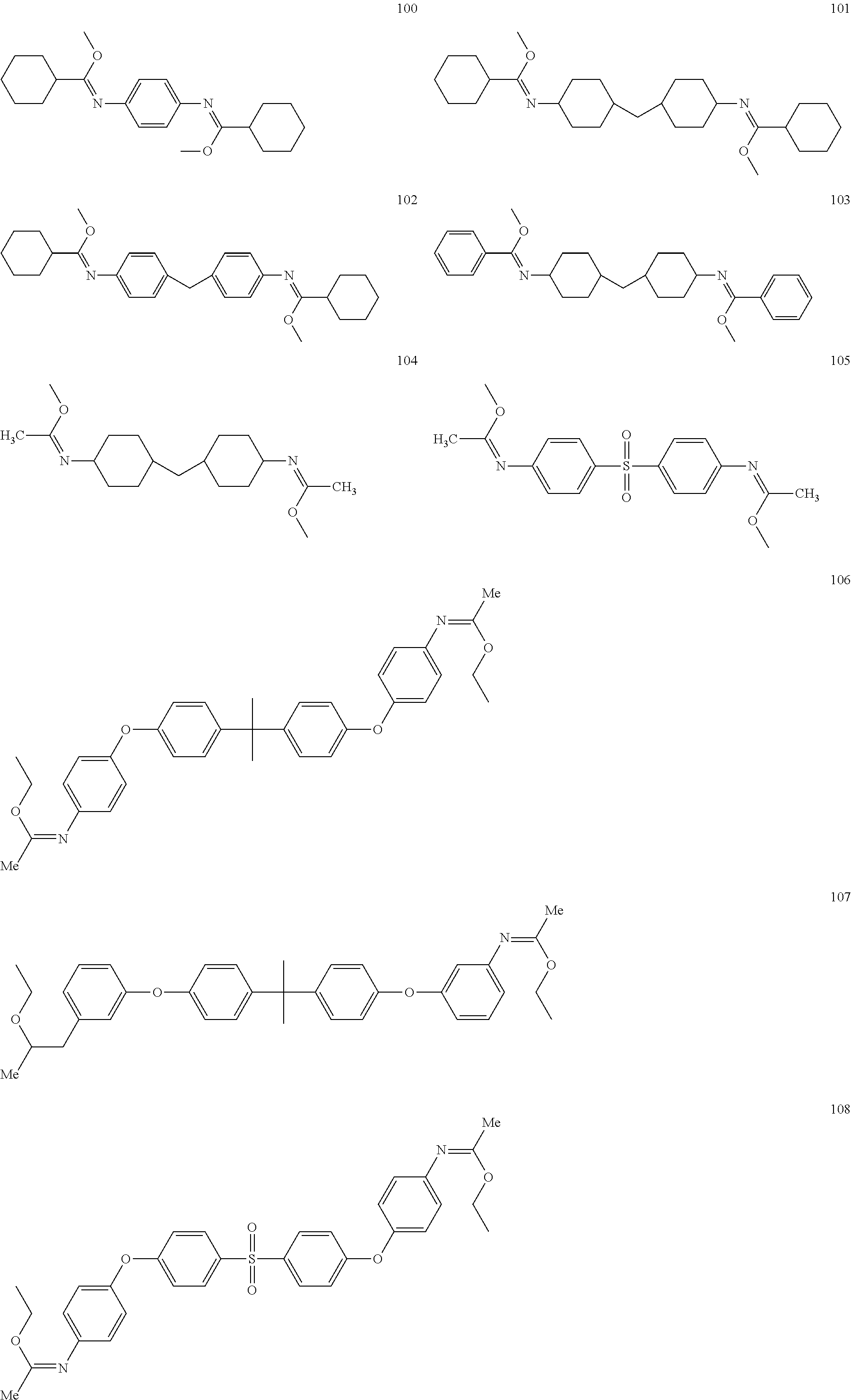

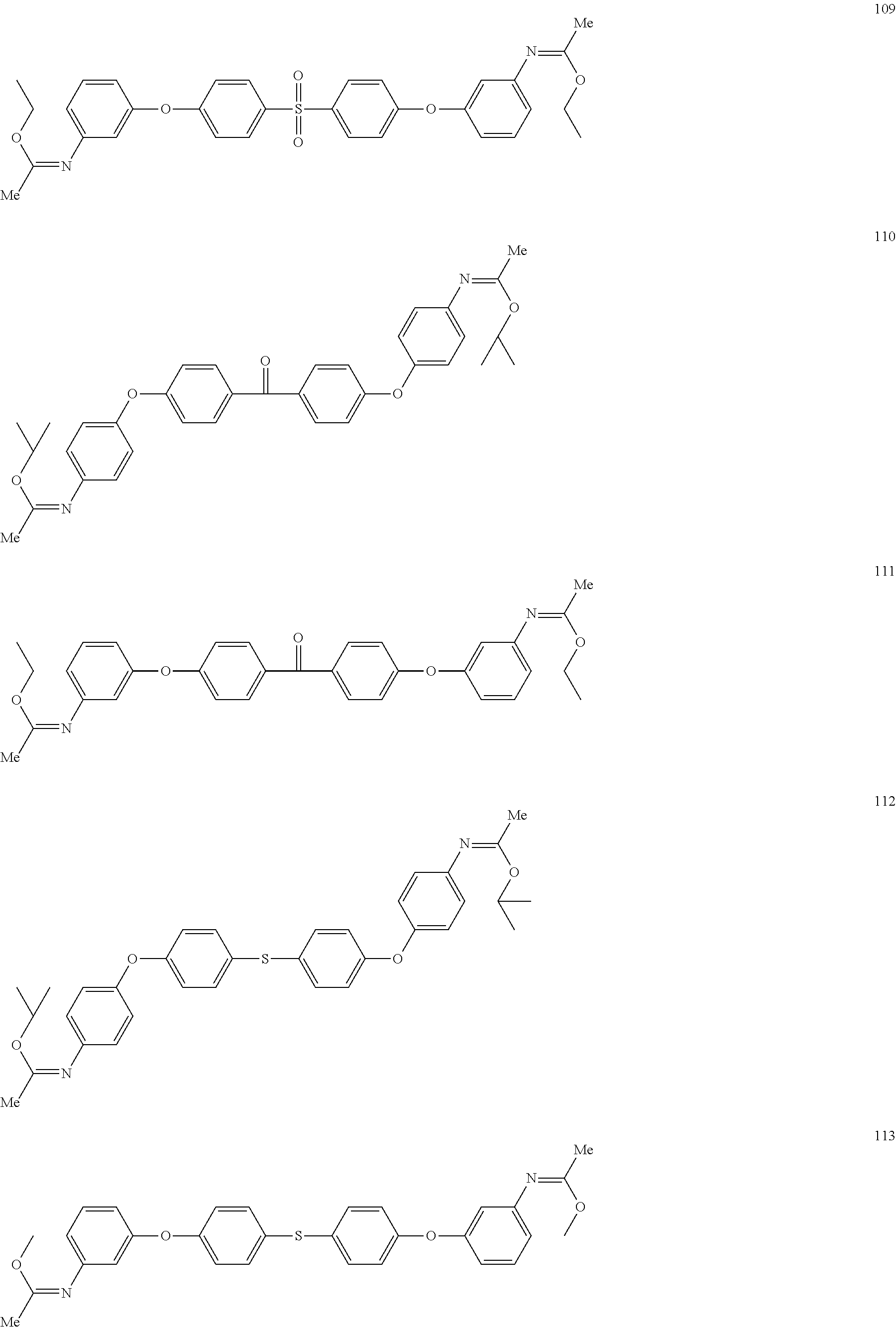

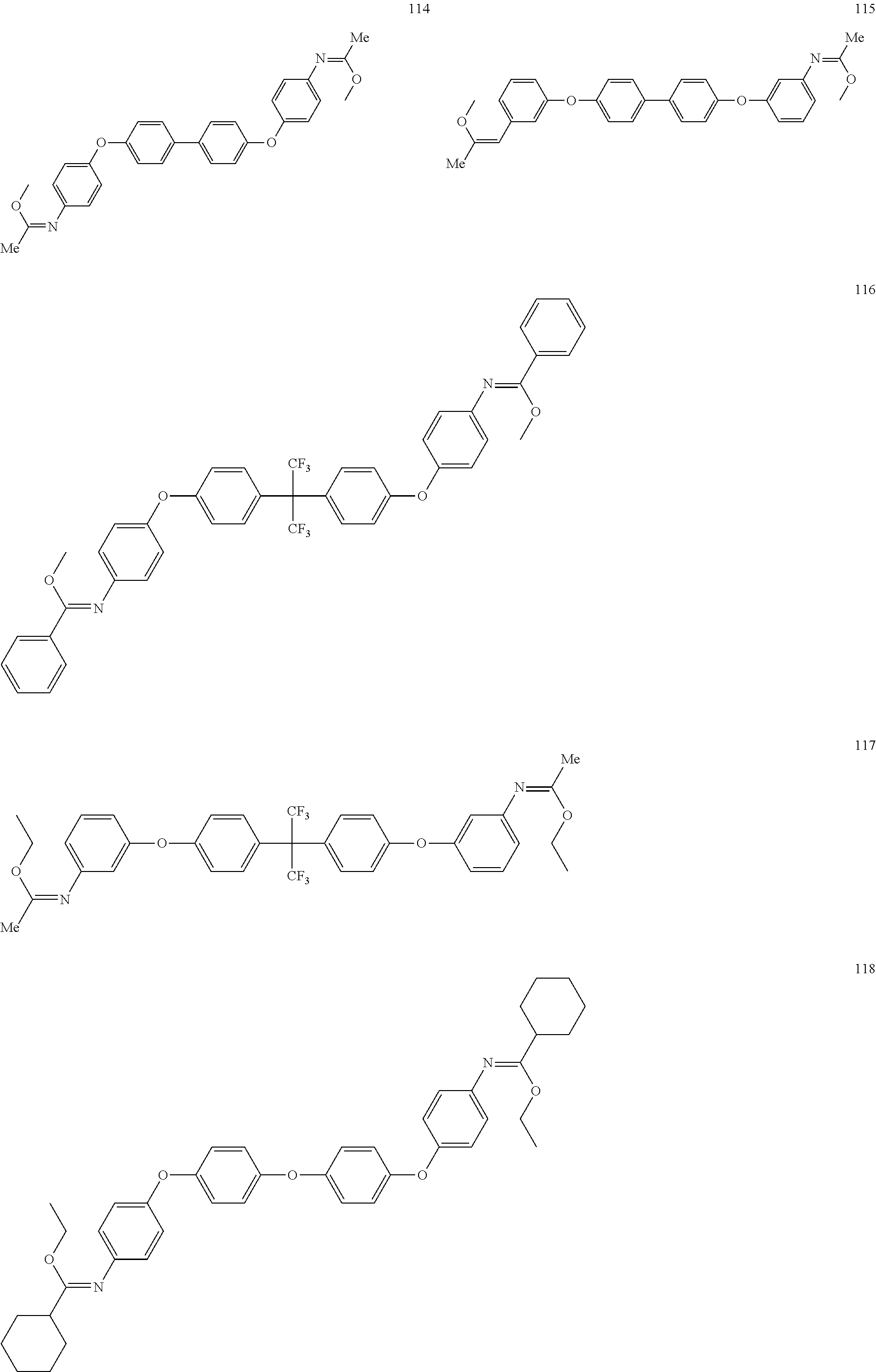

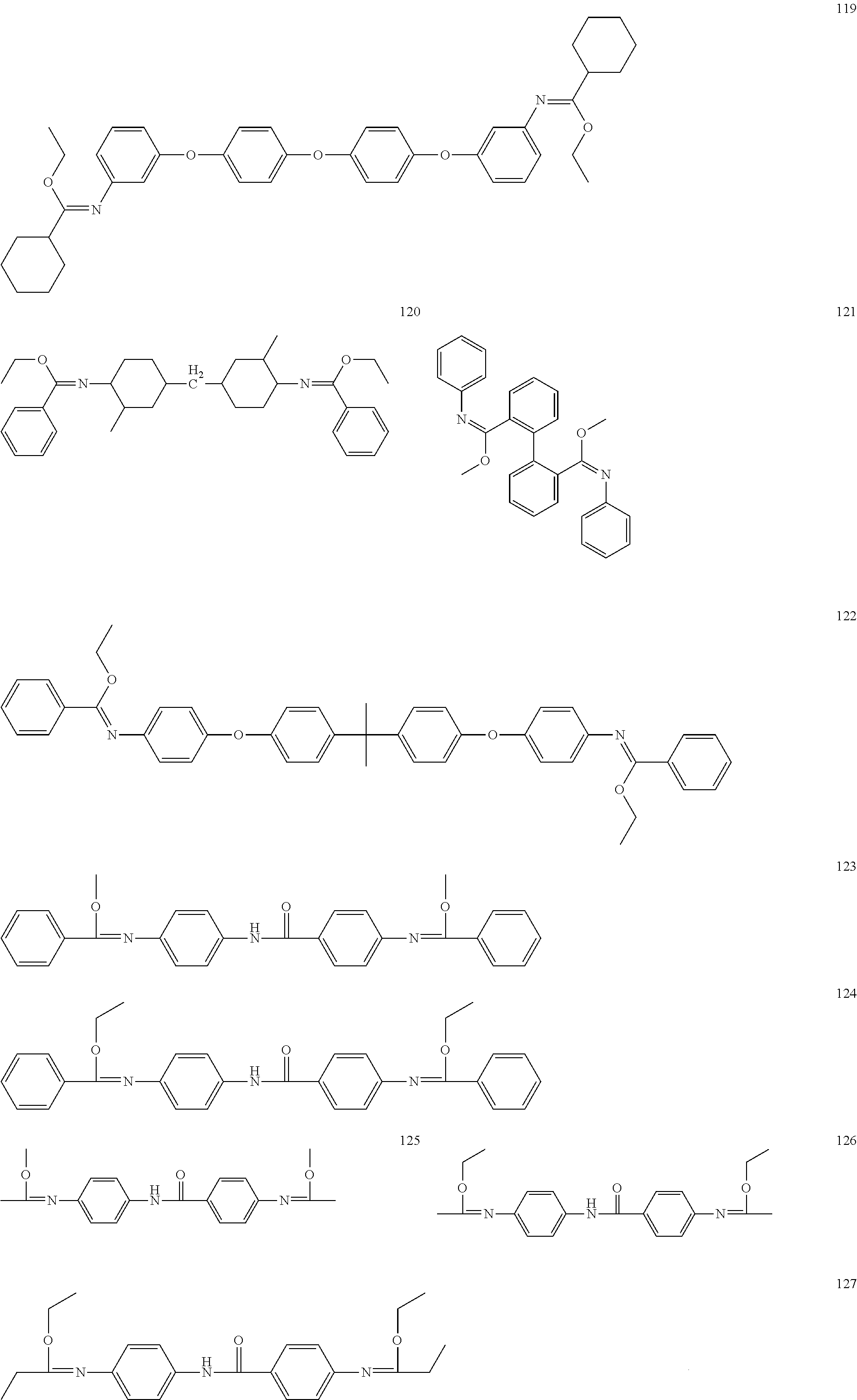

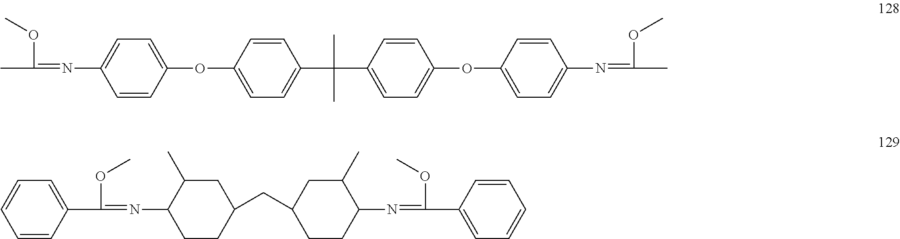

(Chemical Modification Method of Polyester Terminal Carboxyl Group)

Chemical modification of a polyester terminal carboxyl group of the present invention can be carried out by mixing an iminoether compound represented by General Formula (1) and a polyester in the molten state.

In the case where the iminoether compound and the polyester are reacted at a temperature of 100 to 350° C., the iminoether group is reacted with the polyester terminal carboxyl group to generate a carboxylic acid ester as shown in the following reaction scheme. Moreover, because the iminoether compound represented by General Formula (1) becomes an amide compound through the reaction with the polyester terminal carboxyl group, an amide compound is also included in the polyester resin composition. Furthermore, as such, the present invention also relates to a method for producing a carboxylic acid ester.

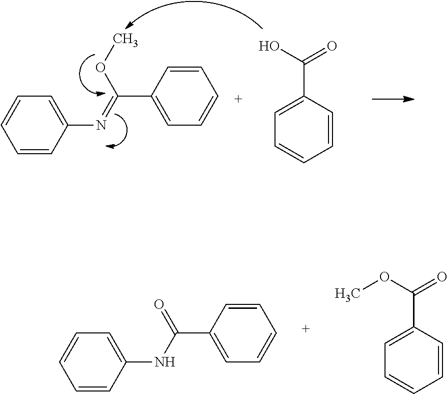

Chemical modification of the polyester terminal carboxyl group can be carried out by performing the reaction of an iminoether compound with a compound having a carboxylic acid group such as polyester at a reaction temperature of 100 to 350° C. The reaction temperature is selected depending on a melting point (Tm) of the polyester to be used, and is preferably (Tm+5)° C. to (Tm+100)° C. and more preferably (Tm+10)° C. to (Tm+80)° C. If the reaction temperature is higher than (Tm+5)° C., the polyester is completely molten, thereby resulting in a good surface state. Moreover, if the reaction temperature is lower than (Tm+100)° C., the polyester becomes to have a good hydrolysis resistance without thermal decomposition.

For example, in the case of polyethylene terephthalate, the reaction temperature is preferably 265 to 360° C. and more preferably 270 to 340° C. In the case of polybutylene terephthalate, the reaction temperature is preferably 230 to 325° C. and more preferably 235 to 305° C. In the case of polyethylene-2,6-naphthalate, the reaction temperature is preferably 270 to 365° C. and more preferably 275 to 345° C. An example satisfying such a temperature range may include subjecting to reaction at 280° C.

The reaction rate of the iminoether compound represented by General Formula (1) with the polyester terminal carboxyl group is preferably 0.1 to 99%, more preferably 1 to 90%, and even more preferably 2 to 80%. By setting the reaction rate to fall within the above-mentioned range, it is possible to sufficiently improve hydrolysis resistance. Furthermore, by setting the reaction rate to fall within the above-mentioned range, it is possible to suppress breakdown of the resulting amide compound from the polyester film, thereby capable of providing better surface state of the polyester film.

Conventionally, esterification of a carboxylic acid has been carried out by the reaction of oxazoline or oxazine with a carboxylic acid. In the case where oxazoline or oxazine is used as a terminal blocking agent, it is known that a ring-opening reaction takes place. Moreover, it is also known that self-condensation proceeds as a side reaction simultaneously with the ring-opening reaction.

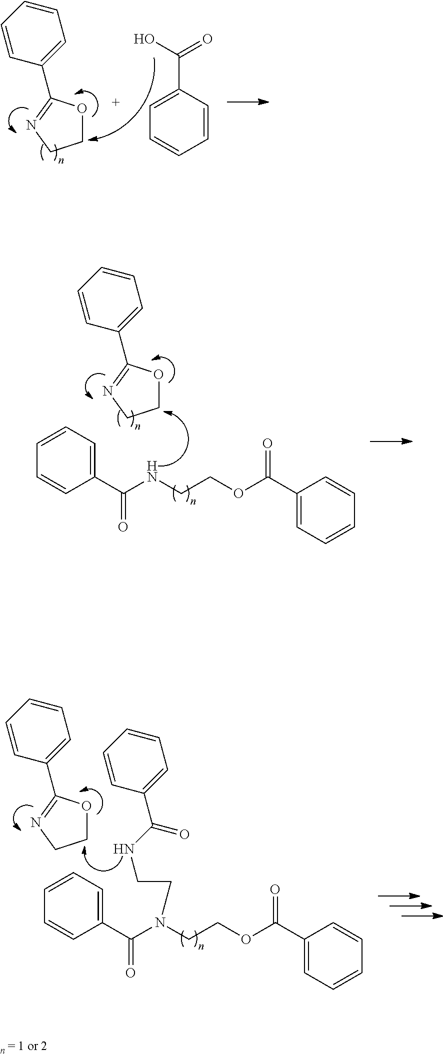

It is believed that self-condensation accompanied with the ring-opening reaction as described above has occurred due to high nucleophilicity of the amide group of the alkyl amide produced by the ring-opening reaction. Meanwhile, it is considered that the iminoether of the present invention exhibits no occurrence of self-condensation because the iminoether of a chain-like compound undergoes no self-condensation and additionally with regard to the iminoether of a cyclic compound, an aromatic amide obtained by an esterification reaction is not high in nucleophilicity thereof. Thus, it is possible to suppress gelation of the iminoether compound in the polyester resin.

(Method for the Synthesis of Iminoether Compound)

As a method for synthesizing the iminoether compound represented by General Formula (1), there have been known a method in which an amide compound is converted into an imidoyl chloride, followed by the reaction with an alkoxide, and a method of reacting an aniline compound with an orthoester compound. As the method for synthesizing the iminoether compound represented by General Formula (1), either method may be employed, but it is preferred to use the method of reacting an aniline compound with an orthoester compound. The polyester resin composition to which the iminoether compound synthesized by the method of reacting an aniline compound with an orthoester compound was added is preferable in terms of having better hydrolysis resistance and color tone. It is believed that the absence of colored substances and reagents deteriorating hydrolysis resistance, and the absence of reaction products in the method of reacting an aniline compound with an orthoester compound contribute to such superior hydrolysis resistance and color tone of the polyester resin. Further, the method of reacting an aniline compound with an orthoester compound is also preferable from the viewpoint that it is possible to synthesize the iminoether compound in a short step. Since the polyester resin composition to which the iminoether compound synthesized by the method of reacting an aniline compound with an orthoester compound was added is superior in hydrolysis resistance and color tone, it is suitably used in a back sheet for a solar cell module. In the case where a less colored polyester resin composition is used in a back sheet for a solar cell module, it is preferred in the viewpoint capable of enhancing the power generation efficiency of the solar cell without lowering the reflectivity of sunlight.

The orthoester compound used in the synthesis of the iminoether compound represented by General Formula (1) is preferably represented by the following General Formula (9).

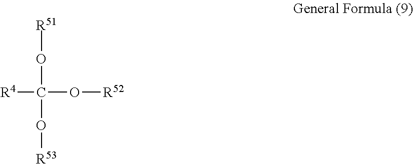

In General Formula (9), R⁴ represents an alkyl group which may have a substituent, a cycloalkyl group which may have a substituent, an aryl group which may have a substituent or an alkoxy group which may have a substituent, and R⁵¹, R⁵² and R⁵³ each independently represent a hydrogen atom, an alkyl group which may have a substituent or an aryl group which may have a substituent.

The orthoester compound used in the synthesis of the iminoether compound represented by General Formula (1) may include, for example, trimethyl orthoacetate, triethyl orthoacetate, tripropyl orthoacetate, tributyl orthoacetate, tribenzyl orthoacetate, trimethyl orthoformate, triethyl orthoformate, tripropyl orthoformate, tributyl orthoformate, tribenzyl orthoformate, trimethyl orthopropionate, triethyl orthopropionate, tripropyl orthopropionate, tributyl orthopropionate, tribenzyl orthopropionate, trimethyl orthobenzoate, triethyl orthobenzoate, tripropyl orthobenzoate, tributyl orthobenzoate, tribenzyl orthobenzoate and the like.

The orthoester compound may be commercially available or synthetic. In the case where it is synthetic, the orthoester compound can be synthesized by a method in which hydrogen cyanide, or a nitrile compound, such as acetonitrile, propionitrile, n-butyronitrile or benzonitrile, is subjected to imidation, followed by the reaction with an alcohol, or by a method of reacting trichlorobenzene with an alkoxide.

(Polyester Resin Composition)

The polyester resin composition of the present invention includes the above-mentioned iminoether compound and a polyester. Within a range not interfering with the effects of the present invention, various additives, for example, a compatibilizer, a plasticizer, a weathering agent, an antioxidant, a thermal stabilizer, a lubricant, an antistatic agent, a brightener, a colorant, a conductive agent, an ultraviolet absorber, a flame retardant, a flame retardant aid, a pigment, and a dye may be added to the polyester resin composition of the present invention.

The polyester is not particularly limited, but is preferably saturated polyester. By using such saturated polyester, it is possible to obtain a polyester film which is excellent from the viewpoint of mechanical strength, as compared to the film using unsaturated polyester.

The polyester has a —COO— bond or a —OCO— bond in one molecular chain of the polymer. In addition, the terminal group of the polyester is preferably a linear saturated polyester synthesized from an aromatic dibasic acid or an ester-forming derivative thereof, a diol or an ester-forming derivative thereof as an OH group, a COOH group, or a protected group thereof (an OR^{X} group, a COOR^{X} group (R^{X} is any substituent such as an alkyl group)). As the linear saturated polyester, for example, those described in JP2009-155479A or JP2010-235824A can be suitably used.

Specific examples of the linear saturated polyester include polyethylene terephthalate (PET), polyethylene isophthalate, polybutylene terephthalate, poly(1,4-cyclohexylenedimethylene terephthalate), and polyethylene-2,6-naphthalate. Among these, polyethylene terephthalate or polyethylene-2,6-naphthalate is particularly preferable, and polyethylene terephthalate is more particularly preferable from the viewpoint of the balance between the mechanical properties and the cost.

The polyester may be a homopolymer or a copolymer. Further, it may be a blend of the polyester with a small amount of any other type of resin, for example, polyimide or the like. It is also possible to use a crystalline polyester which can form anisotropy during the film formation in a melt state as the polyester.

For the molecular weight of the polyester, the weight average molecular weight (Mw) is preferably from 5000 to 100000, more preferably from 8000 to 80000, and particularly preferably from 12000 to 60000, from the viewpoints of heat resistance and viscosity. As the weight average molecular weight of the polyester, a value in terms of polymethyl methacrylate (PMMA), as measured by gel permeation chromatography (GPC) using hexafluoroisopropanol as a solvent, can be used.

The polyester resin composition of the present invention includes the iminoether compound of the present invention in an amount of preferably from 0.05 to 5% by mass, more preferably 0.1 to 4% by mass, even more preferably 0.1 to 2% by mass, and particularly preferably 0.4 to 2% by mass, with respect to the polyester. For example, An example thereof may include the case where the content of the iminoether compound is 1% by mass, or the like. The content of the iminoether compound is preferably the lower limit value described above or higher from the viewpoint of improving hydrolysis resistance when forming a polyester film. Moreover, the content of the iminoether compound is preferably the upper limit value described above or less from the viewpoint of suppressing gelation of the iminoether compound and thereby improving film thickness uniformity when forming a polyester film.

The polyester resin composition of the present invention does not refuse to include a compound other than the above-mentioned iminoether compound as long as it is not contrary to the spirit of the present invention. For example, the polyester resin composition of the present invention can be used in combination with a carbodiimide compound, a ketene imine compound, an epoxy compound, or an oxazoline compound. The iminoether compound of the present invention is preferably 70% by weight or greater, more preferably 80% by weight or greater, and particularly preferably 90% by weight or greater, with respect to an organic compound other than the polyester included in the polyester resin composition of the present invention.

The polyester may be synthesized according to a known method. For example, the polyester may be synthesized by a known polycondensation method, a known ring-opening polymerization method or the like, and any of a transesterification reaction and a reaction by direct polymerization may be applied.

In the case where the polyester used in the present invention is a polymer or a copolymer that is obtained by condensation reaction of an aromatic dibasic acid or an ester-forming derivative thereof and a diol or an ester-forming derivative thereof, as major components, the polyester may be produced by subjecting the aromatic dibasic acid or an ester-forming derivative thereof and the diol or an ester-forming derivative thereof to esterification reaction or transesterification reaction, and then subjecting them to polycondensation reaction. A component derived from a carboxylic acid of the polyester is preferably a component derived from an aromatic dibasic acid or an ester-forming derivative thereof. Further, the carboxyl acid value and the intrinsic viscosity of the polyester may be controlled by selecting the raw materials and the reaction conditions. A polymerization catalyst is preferably added in esterification reaction or transesterification reaction and polycondensation reaction, for effectively performing these kinds of reactions.

As a polymerization catalyst in the polymerization of the polyester, an Al-based, Sb-based, Ge-based, or Ti-based compound is preferably used from the viewpoint of inhibiting the carboxyl group content to a predetermined range or less. Among these, a Ti-based compound is particularly preferred. In the case of using a Ti-based compound, such an embodiment is preferred that the polymerization is carried out by using the Ti-based compound as a catalyst in the range of preferably from 1 ppm to 30 ppm, and more preferably from 3 ppm to 15 ppm. If the proportion of the Ti-based compound is within the range, it is possible to adjust the terminal carboxyl groups to fall within the range as described below, and thus it is possible to keep the hydrolysis resistance of the polymer base low.

In the synthesis of the polyester using a Ti-based compound, for example, the methods described in JP1996-301198B (JP-B08-301198), JP2543624B, JP 3335683B, JP3717380B, JP3897756B, JP3962226B, JP3979866B, JP3996871B, JP4000867B, 4053837B, JP4127119B, JP4134710B, JP4159154B, JP4269704B and JP4313538B, the disclosures of which are incorporated herein by reference, may be applied.

The polyester after the polymerization is preferably subjected to solid-phase polymerization. In this way, a preferred carboxylic acid value may be achieved. The solid-phase polymerization may be in a continuous method (where the resin is filled in a tower, gradually circulated therein with heating for a predetermined period of time, and then discharged) or in a batch method (where the resin is put into a container and heated therein for a predetermined period of time). Specifically, the methods described in JP2621563B, JP3121876B, JP3136774B, JP3603585B, JP3616522B, JP3617340B, JP3680523B, JP3717392B, and JP4167159B, the disclosures of which are incorporated herein by reference, may be applied to the solid-phase polymerization.

The temperature of the solid-phase polymerization is preferably from 170° C. to 240° C., more preferably from 180° C. to 230° C., and even more preferably from 190° C. to 220° C. The time of the solid-phase polymerization is preferably from 5 hours to 100 hours, more preferably from 10 hours to 75 hours, and even more preferably from 15 hours to 50 hours. The solid-phase polymerization is preferably carried out under vacuum or nitrogen atmosphere.

(Polyester Film)

The present invention relates to a polyester film fabricated of the polyester resin composition as described above.

The polyester film of the present invention includes the iminoether compound of the present invention in an amount of preferably from 0.05 to 5% by mass, more preferably 0.1 to 4% by mass, even more preferably 0.1 to 2% by mass, and particularly preferably 0.4 to 2% by mass, with respect to the polyester. For example, An example thereof may include the case where the content of the iminoether compound is 1% by mass, or the like. The content of the iminoether compound is preferably the lower limit value described above or higher from the viewpoint of improving hydrolysis resistance of the polyester film of the present invention. Moreover, the content of the iminoether compound is preferably the upper limit value described above or less from the viewpoint of suppressing gelation of the iminoether compound and thereby improving film thickness uniformity of the polyester film of the present invention. Moreover, by taking the content of the iminoether compound to fall within the above-described range, it is possible to effectively enhance hydrolysis resistance of the polyester film.

The thickness of the polyester film of the present invention varies depending on the uses, but in the case where the polyester film is used as a member of a back sheet for a solar cell module, the polyester film thickness is preferably from 25 μm to 300 μm, and more preferably from 120 μm to 300 μm. When the thickness is 25 μm or more, a sufficient mechanical strength can be obtained, whereas the thickness set to be 300 μm or less is advantageous in terms of costs.

The polyester film of the present invention is preferably stretched, more preferably biaxially stretched, particularly preferably biaxially stretched in plane compared to stretching of a tubular shape, and more particularly preferably sequentially biaxially stretched. The biaxially stretched polyester film is a film which was subjected to stretching in the length direction (MD: Machine Direction) (which is hereinafter also referred to as “longitudinal stretching”) and stretching in the width direction (TD: Transverse Direction) (which is hereinafter also referred to as “transverse stretching”). The longitudinal stretching and the transverse stretching may be carried out once, respectively, or may be carried out in plural times, and the longitudinal stretching and the transverse stretching may be carried out at the same time.

The degree of MD orientation and the degree of TD orientation of the polyester film of the present invention are each preferably 0.14 or more, more preferably 0.155 or more, and particularly preferably 0.16 or more. If each degree of orientation is 0.14 or more, the restriction of the non-crystalline chain is improved (the mobility is lowered), and the hydrolysis resistance is improved. The degree of MD orientation and the degree of TD orientation can be calculated from the degree of MD orientation: Δn(x−z), the degree of TD orientation; Δn(y−z), by measuring the refractive indices in the x, y and z directions of the biaxially oriented film in an atmosphere at 25° C., using an Abbe refractometer, a monochromatic light sodium D-line as a light source, and methylene iodide as a mount solution.

The terminal carboxyl group content in the polyester film (the carboxylic acid value of the polyester, hereinafter also referred to as “AV”) is preferably 25 eq/ton or less, more preferably 20 eq/ton or less, particularly preferably 16 eq/ton or less, and more particularly preferably 15 q/ton or less, with respect to the polyester. If the carboxyl group content is 25 eq/ton or less, the hydrolysis resistance and heat resistance of the polyester film are maintained by the combination with the iminoether compound, and therefore a reduction of strength at the time of wet heat aging can be inhibited low.

In the polyester film of the present invention, the carboxylic acid value after subjecting to storage treatment under conditions of 120° C. and relative humidity of 100% is preferably 200 eq/ton or less, more preferably 100 eq/ton or less, and even more preferably 50 eq/ton or less.

The carboxylic acid value in the polyester can be adjusted by the kind of a polymerization catalyst, the polymerization time, and the film formation conditions (the film formation temperature and time). The carboxylic acid value can be measured by a titration method according to the method described in H. A. Pohl, Anal. Chem. 26 (1954) 2145. Specifically, a polyester is dissolved in benzyl alcohol at 205° C. and a phenol red indicator is added thereto. Then, titration is carried out with a water/methanol/benzyl alcohol solution of sodium hydroxide, and the carboxylic acid value (eq/ton) can be calculated from the titration amount.

The terminal hydroxyl group content in the polyester film is preferably 120 eq/ton or less, and more preferably 90 eq/ton or less, with respect to the polyester. If the hydroxyl group content is 120 eq/ton or less, the reaction between the below-described carbodiimide having a bulky functional group at a specific position and the hydroxyl group is inhibited and thus, the reaction with the carboxyl group is preferentially undergone, which can further reduce the carboxylic acid value. The lower limit of the hydroxyl group content is preferably 20 eq/ton or more from the viewpoint of adhesiveness with an upper layer. The hydroxyl group content in the polyester can be adjusted by the kind of a polymerization catalyst, the polymerization time, or the film formation conditions (the film formation temperature and time). For the terminal hydroxyl group content, a value measured by 1^{H}-NMR, using a deuterated hexafluoroisopropanol solvent, can be used.

In addition, the intrinsic viscosity (IV) of the polyester film of the present invention is preferably 0.55 dl/g to 0.94 dl/g, more preferably 0.60 dl/g to 0.84 dl/g, and particularly preferably 0.70 dl/g to 0.84 dl/g. The intrinsic viscosity of the polyester film is preferably the lower limit value described above or more from the viewpoint of improving film formation properties and therefore improving the film thickness uniformity.

For the intrinsic viscosity (IV) of polyester, in the case where polyester used during the film production is 2 or more types (for example, a case of using the retrieved polyester of JP2011-256337A or the like), the intrinsic viscosity of polyester obtained by mixing all polyester preferably satisfies the above-mentioned range.

For the intrinsic viscosity (IV) of polyester, polyester is dissolved in orthochlorophenol, and from the solution viscosity measured at 25° C., the intrinsic viscosity is obtained according to the following equation.

ηsp/C=[η]+K[η]²·C

Here, ηsp is (solution viscosity/solvent viscosity)−1, C is the dissolved polymer weight per 100 ml of solvent (1 g/100 ml in this measurement), K is a Huggins constant (0.343), and the solution viscosity and the solvent viscosity can be measured using an Ostwald viscometer.

(Production Method of Polyester Film)

(Film Forming Step)

In the film forming step, the melt obtained by melting the polyester and the iminoether compound included in the resin composition for forming the polyester film of the present invention is passed through a gear pump or a filter, then extruded to a cooling roll through a die, and cooled and solidified, whereby a (unstretched) film can be formed. Moreover, the extruded melt can be adhered to the cooling roll using an electrostatic application method. At this time, the surface temperature of the cooling roll can be set to approximately 10° C. to 40° C.

(Stretching Step)

The (unstretched) film formed by the film forming step can be subjected to a stretching treatment in the stretching step. In the stretching step, the (unstretched) film cooled and solidified by the cooling roll is preferably stretched in one or two directions, and more preferably stretched in two directions. With regard to the stretching in two directions (biaxial stretching), the longitudinal stretching and the transverse stretching may be carried out once, respectively, or may be carried out plural times, and the longitudinal stretching and the transverse stretching may be carried out at the same time.

The stretching treatment is carried out preferably at the glass transition temperature (Tg)° C. of the film to (Tg+60)° C., more preferably Tg+3° C. to Tg+40° C., and more preferably at Tg+5° C. to Tg+30° C.

The preferred stretching ratio is from 280% to 500%, more preferably from 300% to 480%, and even more preferably from 320% to 460% in at least one direction. In the case of biaxial stretching, the stretching may be carried out equivalently in the longitudinal and the transverse directions, but it is more preferable that the stretching ratio in one direction is larger than that in the other direction, thereby carrying out inequivalent stretching. Any one of the longitudinal direction (MD) and the transverse direction (TD) may be larger than the other. The stretching ratio as mentioned herein is determined using the following equation.

Stretching ratio (%)=100×(Length after stretching)/(Length before stretching)

The biaxial stretching treatment may be carried out, for example, by a stretching at the glass transition temperature (Tg₁)° C. of a film to (Tg₁+60)° C. in the length direction once or two or more times such that the total ratio is 3- to 6-times and then a stretching at (Tg₁)° C. to (Tg+60)° C. in the width direction such that the ratio is 3- to 5-times.

The biaxial stretching treatment may be carried out by stretching in the length direction using two or more pairs of nip rolls set to a higher peripheral speed on the outlet side (longitudinal stretching), and then by grasping both ends of the film with chucks and extending them in the perpendicular direction (the direction perpendicular to the length direction) (transverse stretching).

In the stretching step, the film can be subjected to a heat treatment before or after the stretching treatment, and preferably after the stretching treatment. By carrying out the heat treatment, fine crystals are produced, which can lead to improvement of the mechanical characteristics or durability. The film may also be subjected to a heat treatment at about 180° C. to 210° C. (more preferably at 185° C. to 220° C.) for 1 second to 60 seconds (more preferably for 2 seconds to 30 seconds).

In the stretching step, the thermal relaxation treatment can be carried out after the heat treatment. The thermal relaxation treatment is a treatment for shrinking the film by applying heat to the film for stress relaxation. The thermal relaxation treatment is preferably carried out in both directions of the MD direction and the TD direction of the film. For the conditions in the thermal relaxation treatment, the treatment is preferably carried out at a temperature lower than the heat treatment temperature, and more preferably at 130° C. to 220° C. Further, for the thermal relaxation treatment, the thermal shrinkage (150° C.) of the film in both of the MD and the TD is preferably from 1% to 12%, and more preferably from 1% to 10%. Furthermore, the thermal shrinkage (150° C.) can be determined according to the following equation, by cutting out a sample having a width of 50 mm at 350 mm in the measurement direction, attaching a target point at an interval of 300 mm near the both ends in the length direction of the sample, fixing one end in an oven adjusted to a temperature of 150° C., leaving the other end to be free for 30 minutes, then measuring the distance between the target points at room temperature, taking this length as L (mm), and using this measured values.

150° C. thermal shrinkage (%)=100×(300−L)/300.

In addition, a case where the thermal shrinkage is positive denotes shrinkage, and a case where the thermal shrinkage is negative denotes stretching.

As described above, a film having excellent hydrolysis resistance and film surface state can be fabricated according to the above-mentioned method. The polyester film of the present invention can be suitably used not only as a protective sheet of a solar cell module (back sheet for a solar cell module) as will be described below, but also in other applications.

In addition, the film of the present invention can also be used as a laminate provided with a coating layer containing at least one functional group selected from COOH, OH, SO₃H, NH₂, or a salt thereof thereon.

(Back Sheet for Solar Cell Module)

The polyester film of the present invention can also be used as a laminate film provided with a coating layer such as a readily adhesive layer thereon. The polyester film or the laminate film of the present invention is widely used for various purposes, and is suitably used as a back sheet for a solar cell module (a protective sheet of a solar cell module). The polyester film of the present invention has excellent adhesiveness and wet heat resistance, and thus, in the case of using the polyester film of the present invention in a back sheet for a solar cell module, a solar cell module can be protected over a long period of time, and therefore the power generation efficiency of the solar cell module is not deteriorated. In addition, since the polyester film of the present invention is suppressed in terms of yellowing, the design characteristics of the solar cell module are also not deteriorated.

By laminating the following functional layers on the polyester film of the present invention, it is possible to form a back sheet for a solar cell module. When laminating functional layers, a readily adhesive layer is preferably provided therebetween. Moreover, before laminating functional layers, the surface of the polyester film is preferably subjected to a surface treatment, and, for example, a flame treatment, a corona treatment, a plasma treatment, or an ultraviolet treatment can be carried out.

<Reflective Layer (Colored Layer)>

A light reflective layer is preferably provided onto the inner side surface (side to be adhered to a sealing material) of the back sheet for a solar cell module of the present invention. By providing a reflective layer, it is possible to return the light to the solar cell by reflecting the light which passes through the solar cell and reaches the back sheet, in sunlight which is incident to the solar cell module. Thus, it is possible to improve the power generation efficiency.

Furthermore, the reflective layer preferably has an adhesive strength of 10 N/cm or greater, and more preferably 20 N/cm or greater, with respect to the sealing material.

(Binder)

As the binder used in the reflective layer, an acryl-based polymer, a polyester-based polymer, a polyurethane-based polymer, a polyolefin-based polymer, or the like can be used, and among these, the polyolefin-based polymer is preferable.

The reflective layer preferably contains a crosslinking agent such as an epoxy-based crosslinking agent, an isocyanate-based crosslinking agent, an oxazoline-based crosslinking agent, or a carbodiimide-based crosslinking agent in order to further improve adhesiveness to the sealing material.

Among these crosslinking agents, the carbodiimide-based crosslinking agent and the oxazoline-based crosslinking agent are particularly preferable from the viewpoint of ensuring adhesiveness after wet heat aging. The carbodiimide-based crosslinking agent used in the present invention is a compound having one or more carbodiimide groups in the molecule thereof.

A white pigment is preferably added to the reflective layer for the purpose of increasing reflectance. Preferred examples of the white pigment may include titanium oxide, barium sulfate, silicon oxide, aluminum oxide, magnesium oxide, calcium carbonate, kaolin, and talc. Among these, titanium oxide is particularly preferable from the viewpoint of whiteness, reflectance, and durability. Although there are three types of crystal systems of rutile, anatase, and brookite in the titanium oxide, titanium oxide having a rutile-type crystalline structure is preferable from the viewpoint of high refractive index, high whiteness, and low photocatalytic activity.

A known additive such as a surfactant or a preservative may be added to the reflective layer, if necessary. Examples of the surfactant may include known surfactants such as an anionic surfactant and a nonionic surfactant. Examples of the anionic surfactant include sodium alkylsulfate and sodium alkylbenzene sulfonate, and examples of the nonionic surfactant include polyoxyethylene alkyl ether. In addition, a fluorine-based surfactant such as sodium perfluoroalkyl sulfate is also preferable.

The thickness of the reflective layer is preferably in the range of 3 μm to 10 μm, and more preferably in the range of 4 μm to 8 μm. When the thickness of the reflective layer is in the range of 3 μm to 10 μm, it is possible to achieve both reflectance and adhesiveness required.

A method of forming the reflective layer is not particularly limited, but known coating methods such as a roll coating method, a bar coater method, a slide-die method, and a gravure coater method can be used. The coating solvent is also not limited, and organic solvent-based solvents such as methyl ethyl ketone, toluene and xylene may be used, or water may be used as a solvent. However, in consideration of low environmental burden, coating using water as a solvent is particularly preferable. These coating solvents may be used alone or may be used in combination thereof. In particular, in the case of water-based coating solvent, the solvent may be used as a mixed solvent obtained by mixing a small amount of a water-miscible organic solvent with water.

Drying of the reflective layer is also not particularly limited, but drying is preferably carried out at a temperature of about 120° C. to 200° C. for about 1 minute to 10 minutes from the viewpoint of shortening the drying time. In the case where the drying temperature is lower than 120° C., drying time becomes longer, and thus, this is disadvantageous in production thereof. In contrast, In the case where the drying temperature is higher than 200° C., the flatness of the obtained back sheet is impaired in some cases.

<Overcoat Layer>

For the purposes of improving adhesiveness to the sealing material, an overcoat layer may be provided on the reflective layer in the back layer for a solar cell module of the present invention.

As the binder of the overcoat layer, those described in the above “Reflective Layer” section can be preferably used.

As the type of the crosslinking agent of the overcoat layer, those described in the above “Reflective Layer” section can be preferably used. The content of the crosslinking agent of the overcoat layer is preferably 5% by mass to 40% by mass, and more preferably 10% by mass to 30% by mass, with respect to the binder constituting the overcoat layer. When the content of the crosslinking agent is 5% by mass or greater, it is possible to obtain a sufficient crosslinking effect while keeping the strength and the adhesiveness of the overcoat layer, and when the content of the crosslinking agent is 40% by mass or less, the pot life of the coating liquid can be maintained longer.

As the types of and the amounts added of other additives in the overcoat layer, those described in the above “Reflective Layer” section can be preferably used.

The film thickness of the overcoat layer is preferably in the range of 0.1 μm to 1.0 μm, and more preferably in the range of 0.2 μm to 0.8 μm. When the thickness of the overcoat layer is in the range of 0.1 μm to 1.0 μm, it is possible to obtain high adhesiveness to the sealing material.

As the coating method, the coating solvent, and the drying method of the overcoat layer, those or the method described in the above “Reflective Layer” section can be preferably used.

<Rear Surface Layer>

The back sheet for a solar cell module of the present invention is preferably provided with a rear surface layer for protecting a support on the outer surface (the surface of the opposite side of a solar cell). As the binder of the rear surface layer, the silicone-based complex polymers described below are preferably used from the viewpoint of durability and adhesiveness to the support. The silicone-based complex polymer (hereinafter, sometimes referred to as “complex polymer”) is a polymer including a —(Si(R¹)(R²)—O)ₙ— moiety and a polymer structure moiety which is copolymerized with the —(Si(R¹)(R²)—O)ₙ— moiety in the molecule thereof. By using the silicone-based complex polymer as the binder of the rear surface layer, it is possible to make the adhesiveness between the rear surface layer and the support particularly favorable, and therefore it is possible to keep a reduction of the adhesiveness low over a long period of time.

The silicone-based complex polymer is preferably in the form of a water-based polymer dispersion (so-called latex). The preferred particle diameter of latex of the silicone-based complex polymer is about 50 nm to 500 nm, and the preferred concentration thereof is about 15% by mass to 50% by mass.

In the case where the water-based polymer is in the form of latex, the silicone-based complex polymer preferably has a functional group with water affinity, such as a carboxyl group, a sulfonic acid group, a hydroxyl group, or an amide group. In the case where the silicone-based complex polymer has a carboxyl group, the carboxyl group may be neutralized with sodium, ammonium, or amine.

In addition, in the case where the silicone-based complex polymer is used in the form of latex, an emulsion stabilizer such as a surfactant (example: anionic or nonionic surfactant) or a polymer (example: polyvinyl alcohol) may be contained in order to improve the stability. Furthermore, if necessary, known compounds as an additive in latex may be added such as a pH adjusting agent (example: ammonia, triethylamine, or sodium hydrogen carbonate), a preservative (example: 1,3,5-hexahydro-(2-hydroxyethyl)-s-triazine, or 2-(4-thiazolyl)benzimidazole), a thickening agent (example: sodium polyacrylate, or methyl cellulose), and a film-forming aid (example: butyl carbitol acetate).

A crosslinking agent is preferably added to the rear surface layer in order to improve the adhesiveness to the support. As the type of the crosslinking agent, those described in the above “Reflective Layer” section can be used.

The content of the crosslinking agent is preferably 5% by mass to 40% by mass, and more preferably 10% by mass to 30% by mass, with respect to the binder constituting the rear surface layer. When the content of the crosslinking agent is 5% by mass or greater, it is possible to obtain a sufficient crosslinking effect while keeping the adhesiveness to the support, and when the content of the crosslinking agent is 40% by mass or less, the pot life of the coating liquid can be maintained longer.

An ultraviolet absorber is preferably added to the rear surface layer. In the case of an organic-based ultraviolet absorber, examples of the ultraviolet absorber may include ultraviolet absorbers such as a salicylic acid-based absorber, a benzophenone-based absorber, a benzotriazole-based absorber, and a cyanoacrylate-based absorber and ultraviolet stabilizers such as a hindered amine-based stabilizer. In addition, examples of an inorganic-based ultraviolet absorber may include metal oxides such as titanium oxide, zinc oxide, and cerium oxide, and carbon-based components such as carbon, fullerene, a carbon fiber, and a carbon nanotube. Among these, titanium oxide is particularly preferable from the viewpoint of cost and durability.

Although the amount of ultraviolet absorber added to the rear surface layer varies depending on the type of the ultraviolet absorber, the amount is preferably in the range of 0.2 g/m² to 5 g/m², and more preferably in the range of 0.3 g/m² to 3 g/m².

A white pigment may be added to the rear surface layer for the purpose of compensating for the reflectance of the reflective layer. As the type of the white pigment, the white pigments described in the above “Reflective Layer” section can be preferably used. The amount of the white pigment added to the rear surface layer is preferably in the range of 0.3 g/m² to 10 g/m², and more preferably in the range of 4 g/m² to 9 g/m². When the amount added is 0.3 g/m² to 10 g/m², it is possible to achieve both excellent adhesiveness and reflectance improvement. Moreover, in the case of using titanium oxide as the white pigment, it is possible for titanium oxide to serve as both a pigment and an ultraviolet absorber. As the types of and the amounts of other additives added in the rear surface layer, those described in the above “Reflective Layer” section can be preferably used.

The thickness of the rear surface layer is preferably in the range of 3 μm to 12 μm, and more preferably in the range of 4 μm to 8 μm. When the thickness of the rear surface layer is in the range of 3 μm to 12 μm, it is possible to achieve both durability and adhesiveness required.

As the forming method, the coating solvent, and the drying method of the rear surface layer, those or the method described in the above “Reflective Layer> can be preferably used.

<Rear Surface Protective Layer>

For the purposes of further improving durability, a rear surface protective layer may be provided on the rear surface layer in the back sheet for a solar cell module of the present invention.

The binder of the rear surface protective layer is preferably a fluorine-based polymer from the viewpoint of durability. The fluorine-based polymer capable of being preferably used in the present invention is a polymer including a fluorine-containing monomer in the main chain or side chain thereof. Although the fluorine-containing monomer may be included in any one of the main chain or side chain, the fluorine-containing monomer is preferably included in the main chain from the viewpoint of durability.

In the case of using the fluorine-based polymer in the form of latex, the particle diameter of the fluorine-based polymer is preferably about 50 nm to 500 nm, and the solid content concentration is preferably about 15% by mass to 50% by mass. In the case where the water-based polymer is in the form of latex, the fluorine-based polymer preferably has a functional group with water affinity, such as a carboxyl group, a sulfonic acid group, a hydroxyl group, or an amide group.

A crosslinking agent is preferably added to the rear surface protective layer in order to improve the adhesiveness to the support. As the type of the crosslinking agent, those described in the above “Reflective Layer” section can be used.

A slipping agent may be added to the rear surface protective layer, if necessary. Examples of the slipping agent may include a synthetic wax-based compound, a natural wax-based compound, a surfactant-based compound, an inorganic compound, and an organic resin-based compound. Among these, from the viewpoint of the surface strength of the polymer layer, a compound selected from a synthetic wax-based compound, a natural wax-based compound and a surfactant-based compound is preferable.

Colloidal silica may be added to the rear surface protective layer, if necessary. The colloidal silica is colloidal silica in which fine particles having silicon oxide as a main component are present in a fine particle state in water, monohydric alcohols, diols, or a mixture thereof as a dispersion medium.

As the types of and the amounts added of other additives in the rear surface protective layer, those described in the above “Reflective Layer” section can be preferably used.

The thickness of the rear surface protective layer is preferably in the range of 0.5 μm to 6 μm, and more preferably in the range of 1 μm to 5 μm. When the thickness of the rear surface protective layer is 0.5 μm or greater, durability is sufficient, and when the thickness is 6 μm or less, it is advantageous in terms of cost. As the coating method, the coating solvent, and the drying method of the rear surface protective layer, those or the method described in the above “Reflective Layer” section can be preferably used.

(Solar Cell Module)

The solar cell module of the present invention includes the polyester film of the present invention or the back sheet for a solar cell module of the present invention.

The solar cell module of the present invention is configured such that a solar cell element that converts the light energy of sunlight into electrical energy is disposed between a transparent substrate, on which sunlight is incident, and the polyester film (back sheet for a solar cell) of the present invention described above. The space between the substrate and the polyester film can be configured to be sealed with a resin (a so-called sealing material) such as an ethylene-vinyl acetate copolymer.

Members other than the solar cell module, the solar cell, and the back sheet are described in detail in, for example, “Constituent Materials for Solar Power Generation System” (under the supervision of Eiichi Sugimoto, published by Kogyo Chosakai Publishing Co., Ltd., 2008).

The transparent substrate may have optical transparency by which sunlight can be transmitted, and can be suitably selected from base materials that transmit light. From the viewpoint of power generation efficiency, a base material having higher light transmittance is preferable. As such a substrate, for example, a glass substrate, a substrate of a transparent resin such as an acrylic resin, or the like can be suitably used.

As the solar cell element, various known solar cell elements can be applied such as silicon-based elements such as single crystal silicon, polycrystalline silicon, and amorphous silicon; and Group III-V or Group II-VI compound semiconductor-based elements such as copper-indium-gallium-selenium, copper-indium-selenium, cadmium-tellurium, and gallium-arsenic.

### EXAMPLES

Hereinafter, features of the present invention will be more specifically described with reference to Examples and Comparative Examples. The materials, amounts used, proportions, treatment contents, treatment procedures, and the like indicated in the Examples below may be suitably modified within a range not departing from the spirit of the present invention. Therefore, the scope of the present invention is not to be interpreted as limiting to the specific examples shown below. Unless otherwise specifically indicated, the “part(s)” is on the basis of mass.

As an iminoether compound, the following compounds were used in Examples.

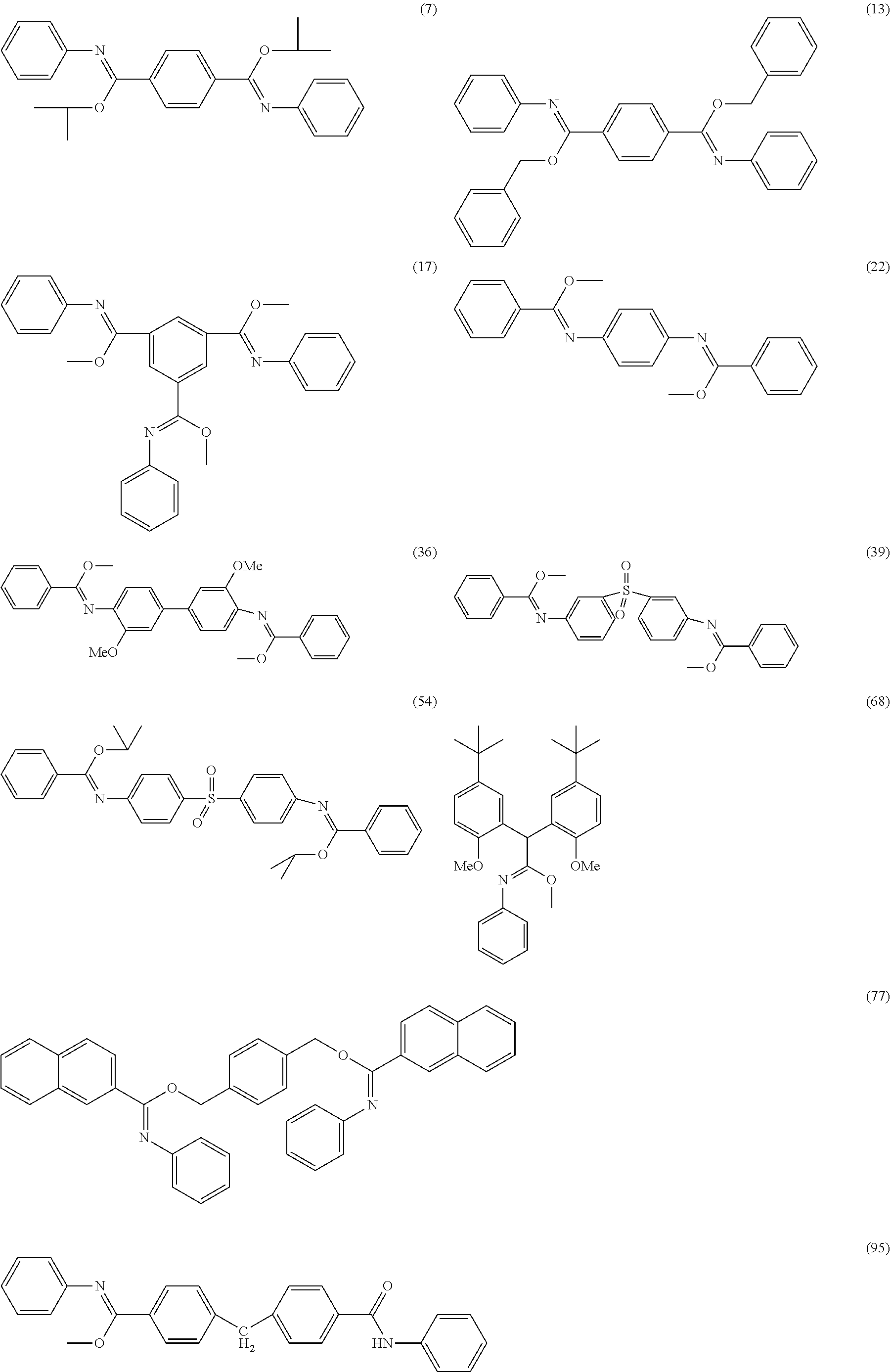

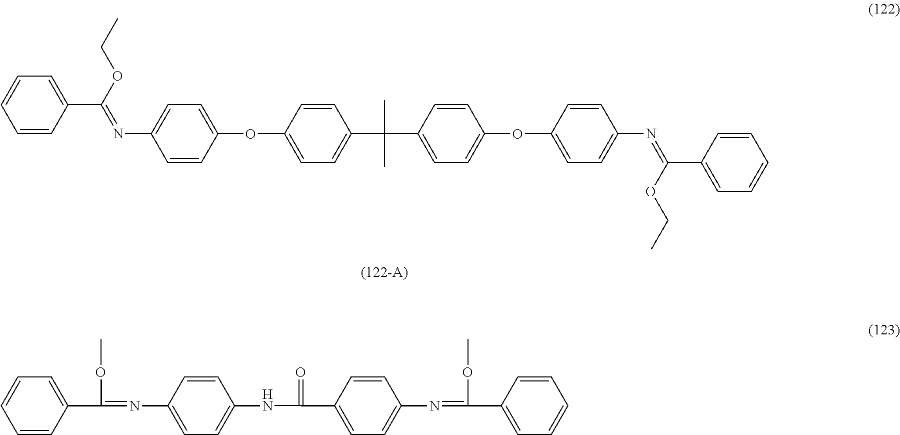 [SMILES: CCO/C(=N\c1ccc(Oc2ccc(C(C)(C)c3ccc(Oc4ccc(/N=C(\OCC)c5ccccc5)cc4)cc3)cc2)cc1)c1ccccc1 ; COC(=Nc1ccc(NC(=O)c2ccc(N=C(OC)c3ccccc3)cc2)cc1)c1ccccc1]

### Synthesis Example 1

### Synthesis of Exemplary Compound 1

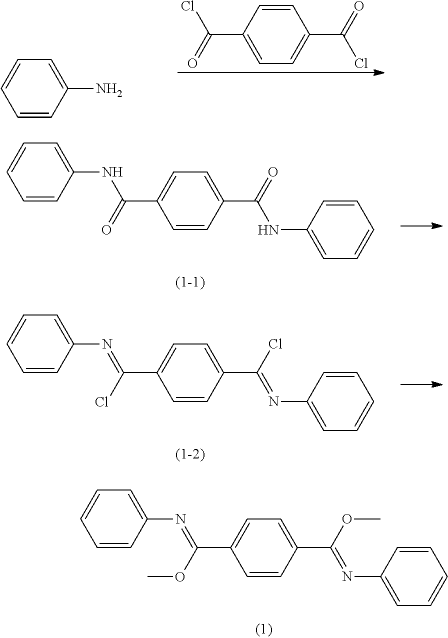

To a solution of 100 g (1.1 mol) of aniline in 500 ml of N,N-dimethylacetamide was added portionwise under ice-cooling 99 g (0.49 mol) of terephthalic acid dichloride in 10 divided portions. The mixture was allowed to warm to room temperature and stirred for 10 hours. Thereafter, the reaction solution was slowly added to 5 L of water to precipitate a solid. The solid was separated by filtration, and the resulting solid was dispersed in water and was subjected to filtration again. This operation was repeated twice. The resulting solid was dried to give 136 g (88%) of (1-1).

A suspension of 32 g (0.1 mol) of (1-1) in 400 ml of thionyl chloride and 0.3 ml of N,N-dimethylformamide was stirred for 11 hours under heating to reflux. Then, 0.3 ml of N,N-dimethylformamide was added thereto, followed by stirring for another 4 hours, and therefore the reaction system was completely dissolved. Thereafter, thionyl chloride was distilled off and 300 ml of hexane was added to precipitate a solid. The solid was collected by filtration to give 35 g (quant.) of (1-2).

150 ml of a solution of 35 g (0.1 mol) of (1-2) in tetrahydrofuran was cooled to −5° C., and 42.5 g (0.22 mol) of a 28% solution of sodium methoxide in methanol was added dropwise thereto. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, crystallization in ethyl acetate was carried out to give 30 g (87%) of Exemplary Compound (1). The resulting Exemplary Compound was identified by ¹H-NMR.

### Synthesis Example 2

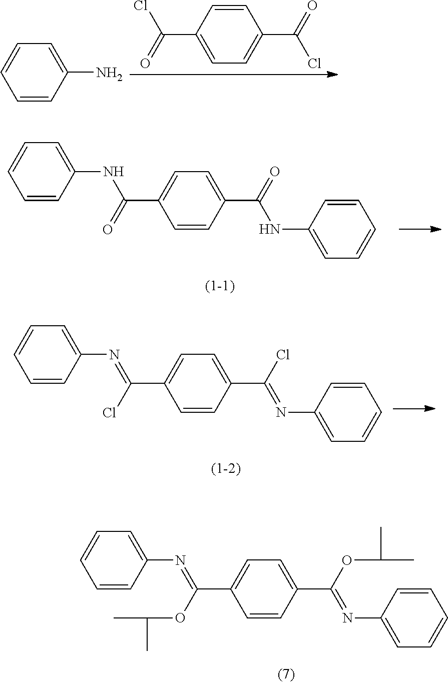

8.8 g (0.22 mol) of sodium hydride (60%) was dispersed in hexane, and after allowing to stand, the supernatant hexane was removed by a Pasteur pipette. This operation was repeated twice. 150 ml of a tetrahydrofuran solution was added thereto. Under ice-cooling, 14 g (0.23 mol) of isopropanol was added dropwise. Completion of bubble generation from the system was visually confirmed, and a solution of sodium isopropoxide in tetrahydrofuran was prepared.

150 ml of a solution of 35 g (0.1 mol) of (1-2) in tetrahydrofuran was cooled to −5° C., and the prepared tetrahydrofuran solution of sodium isopropoxide was added dropwise thereto. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, crystallization in an ethyl acetate/hexane mixed solvent was carried out to give 33 g (83%) of Exemplary Compound (7). The resulting Exemplary Compound was identified by ¹H-NMR.

### Synthesis Example 3

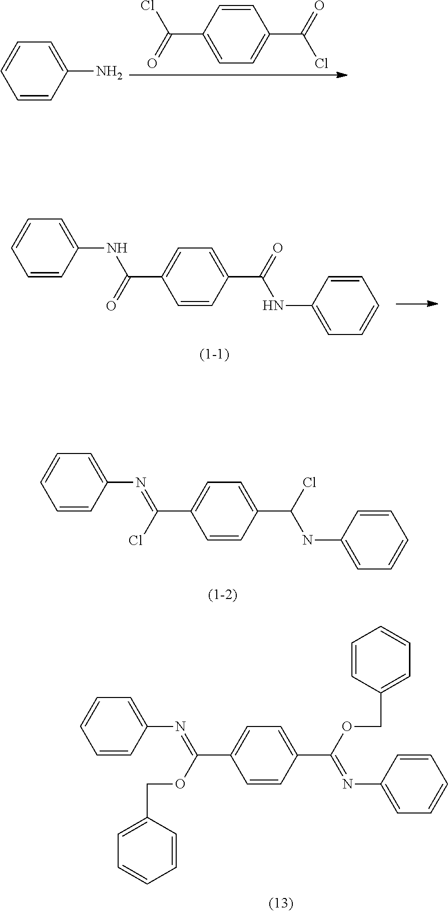 [SMILES: Nc1ccccc1 ; O=C(Cl)c1ccc(C(=O)Cl)cc1 ; O=C(Nc1ccccc1)c1ccc(C(=O)Nc2ccccc2)cc1 ; Cl/C(=N\c1ccccc1)c1ccc(C(Cl)Nc2ccccc2)cc1 ; c1ccc(CO/C(=N\c2ccccc2)c2ccc(/C(=N/c3ccccc3)OCc3ccccc3)cc2)cc1]

8.8 g (0.22 mol) of sodium hydride (60%) was dispersed in hexane, and after allowing to stand, the supernatant hexane was removed by a Pasteur pipette. This operation was repeated twice. 150 ml of a tetrahydrofuran solution was added thereto. Under ice-cooling, 25 g (0.23 mol) of benzyl alcohol was added dropwise thereto. Completion of bubble generation from the system was visually confirmed, and a solution of sodium benzyl oxide in tetrahydrofuran was prepared.

150 ml of a solution of 35 g (0.1 mol) of (1-2) in tetrahydrofuran was cooled to −5° C., and the prepared tetrahydrofuran solution of sodium benzyl oxide was added dropwise thereto. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, crystallization in an ethyl acetate/hexane mixed solvent was carried out to give 40 g (81%) of Exemplary Compound (13). The resulting Exemplary Compound was identified by ¹H-NMR.

### Synthesis Example 4

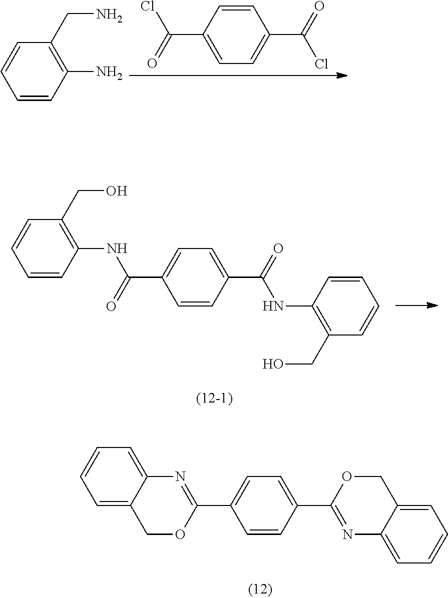 [SMILES: NCc1ccccc1N ; O=C(Cl)c1ccc(C(=O)Cl)cc1 ; O=C(Nc1ccccc1CO)c1ccc(C(=O)Nc2ccccc2CO)cc1 ; c1ccc2c(c1)COC(c1ccc(C3=Nc4ccccc4CO3)cc1)=N2]

To a solution of 24.6 g (0.2 mol) of 2-aminobenzyl alcohol in 200 ml of N,N-dimethylacetamide was added portionwise under ice-cooling 20.3 g (0.1 mol) of terephthalic acid dichloride. The temperature of the reaction system was allowed to warm to room temperature, followed by stirring for 3 hours. The reaction solution was slowly added to 3 L of water to precipitate a solid. The solid was separated by filtration, and the resulting solid was dispersed in water and was subjected to filtration again. This operation was repeated twice. The resulting solid was dried to give 36 g (96%) of (12-1).

5.2 g (13.8 mmol) of (12-1) was warmed to 250° C. and stirred for 3 hours. After cooling, the reaction solution was diluted with ethyl acetate and purified by silica gel column chromatography with an ethyl acetate/hexane mixed solvent. The solvent was distilled off to give 1.6 g (35%) of Exemplary Compound (12). The resulting Exemplary Compound was identified by ¹H-NMR.

### Synthesis Example 5

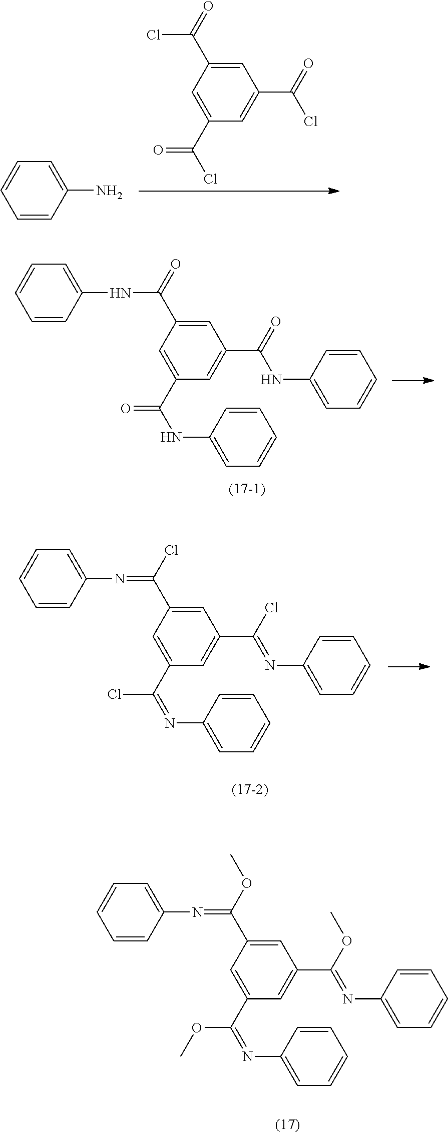 [SMILES: Nc1ccccc1 ; O=C(Cl)c1cc(C(=O)Cl)cc(C(=O)Cl)c1 ; O=C(Nc1ccccc1)c1cc(C(=O)Nc2ccccc2)cc(C(=O)Nc2ccccc2)c1 ; ClC(=Nc1ccccc1)c1cc(/C(Cl)=N/c2ccccc2)cc(/C(Cl)=N\c2ccccc2)c1 ; COC(=Nc1ccccc1)c1cc(/C(=N/c2ccccc2)OC)cc(/C(=N\c2ccccc2)OC)c1]

To a solution of 70 g (0.75 mol) of aniline and 59 g (0.75 mol) of pyridine in 500 ml of N,N-dimethylacetamide was added dropwise under ice-cooling a solution of 50 g (0.19 mol) of trimesic acid trichloride in 100 ml of N,N-dimethylacetamide. After the reaction system was allowed to warm to room temperature, the mixture was stirred for 3 hours. The reaction solution was slowly added to 5 L of water to precipitate a solid. The solid was separated by filtration. The resulting solid was dispersed in methanol and filtered again. This operation was repeated twice. The resulting solid was dried to give 80 g (97%) of (17-1).

A suspension of 21.8 g (50 mmol) of (17-1) in 100 ml of thionyl chloride was stirred for 3 hours under heating to reflux. It was confirmed that the reaction system became clear, followed by stirring for another 2 hours. Thionyl chloride was distilled off to give (17-2). 200 ml of tetrahydrofuran was added, and under ice-cooling, 21.2 g (110 mol) of a 28% solution of sodium methoxide in methanol was added dropwise. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, purification was carried out by silica gel column chromatography using an ethyl acetate/hexane mixed solvent as an eluent. The solvent was distilled off to give 21 g (88%) of Exemplary Compound (17). The resulting Exemplary Compound was identified by ¹H-NMR.

### Synthesis Example 5

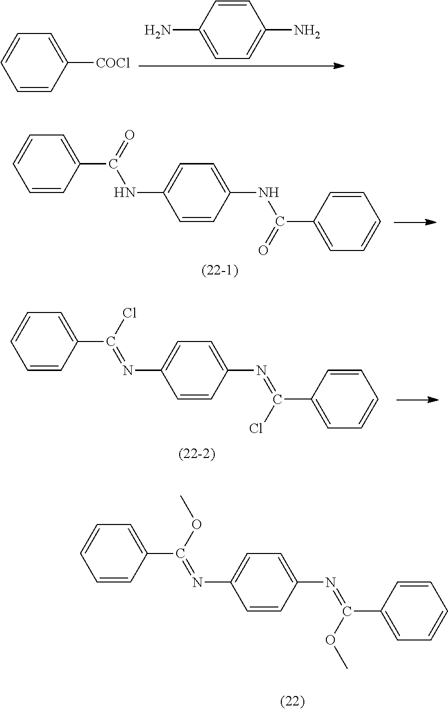

To a solution of 65 g (0.6 mol) of para-phenylenediamine and 95 g (1.2 mol) of pyridine in 400 ml of N,N-dimethylacetamide was added dropwise under ice-cooling 154 ml (1.32 mol) of benzoyl chloride. The reaction system was allowed to warm to room temperature, followed by stirring for 5 hours. After 50 ml of methanol was added, the reaction solution was slowly added to 5 L of water to precipitate a solid. The solid was separated by filtration. The resulting solid was dispersed in methanol and filtered again. This operation was repeated twice. The resulting solid was dried to give 173 g (91%) of (22-1).

A suspension of 32 g (0.1 mol) of (22-1) in 150 ml of thionyl chloride was stirred for 6 hours under heating to reflux. The complete dissolution of the reaction system was confirmed, followed by stirring for another 2 hours. Thereafter, thionyl chloride was distilled off and 300 ml of hexane was added to precipitate a solid. The solid was collected by filtration to give 35 g (quant.) of (22-2).

150 ml of a solution of 35 g (0.1 mol) of (22-2) in tetrahydrofuran was cooled to −5° C., and 42.5 g (0.22 mol) of a 28% solution of sodium methoxide in methanol was added dropwise thereto. The temperature of the system was allowed to warm to room temperature, followed by stirring for 10 minutes, and ethyl acetate/tetrahydrofuran/water was added to carry out liquid separation. The organic phase was washed with water, and dried over magnesium sulfate. After distilling off the solvent, crystallization in ethyl acetate was carried out to give 30 g (87%) of Exemplary Compound (22). The resulting Exemplary Compound was identified by ¹H-NMR.

### Synthesis Example 6

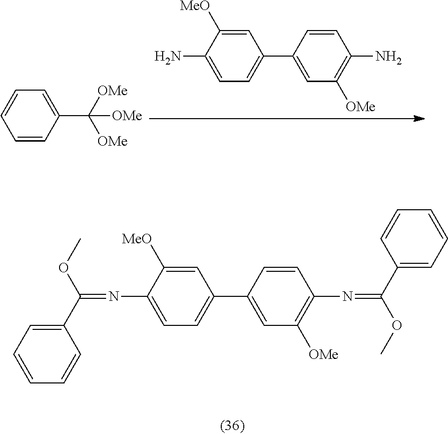 [SMILES: COC(OC)(OC)c1ccccc1 ; COc1cc(-c2ccc(N)c(OC)c2)ccc1N ; COC(=Nc1ccc(-c2ccc(N=C(OC)c3ccccc3)c(OC)c2)cc1OC)c1ccccc1]

To 8 g (44 mmol) of trimethyl orthobenzoate and 4.9 g (20 mmol) of orthomethoxy benzidine in 25 ml of toluene was added a catalytic amount of a para-toluenesulfonic acid monohydrate, and the mixture was stirred for 5 hours under heating to reflux. Methanol, which was from reflux at the reaction system temperature of 100° C. or lower, was removed by the Dean-Stark apparatus. After confirming the completion of the reaction by TLC, ethyl acetate/water was added to carry out liquid separation. The organic phase was washed with water, and dried over magnesium sulfate. After distilling off the solvent, crystallization in an ethyl acetate/hexane mixed solvent was carried out to give 9 g (94%) of Exemplary Compound (36). The resulting Exemplary Compound was identified by ¹H-NMR.

### Synthesis Example 7

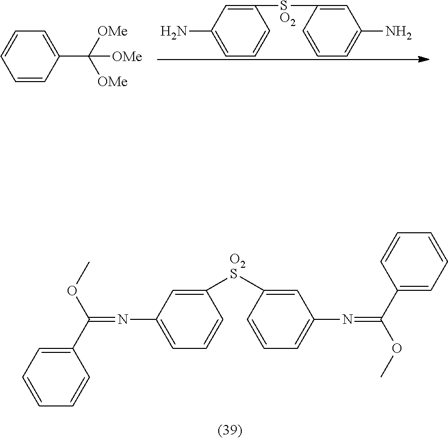 [SMILES: COC(OC)(OC)c1ccccc1 ; Nc1cccc(S(=O)(=O)c2cccc(N)c2)c1 ; COC(=Nc1cccc(S(=O)(=O)c2cccc(N=C(OC)c3ccccc3)c2)c1)c1ccccc1]

To 20 g (44 mmol) of trimethyl orthobenzoate and 11.4 g (46 mmol) of 3,3-diaminodiphenyl sulfone in 75 ml of toluene was added a catalytic amount of a para-toluenesulfonic acid monohydrate, and the mixture was stirred for 5 hours under heating to reflux. Methanol, which was from reflux at the reaction system temperature of 100° C. or lower, was removed by the Dean-Stark apparatus. After confirming the completion of the reaction by TLC, ethyl acetate/water was added to carry out liquid separation. The organic phase was washed with water, and dried over magnesium sulfate. After distilling off the solvent, purification was carried out by silica gel column chromatography using an ethyl acetate/hexane mixed solvent as an eluent. The solvent was distilled off to give 20 g (89%) of Exemplary Compound (39). The resulting Exemplary Compound was identified by ¹H-NMR.

### Synthesis Example 8

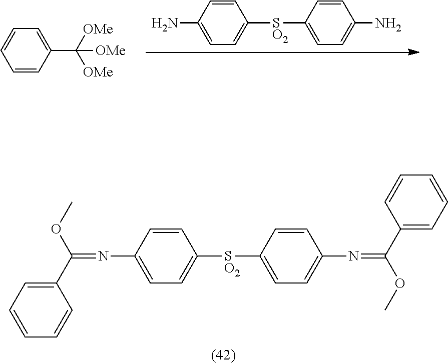 [SMILES: COC(OC)(OC)c1ccccc1 ; Nc1ccc(S(=O)(=O)c2ccc(N)cc2)cc1 ; COC(=Nc1ccc(S(=O)(=O)c2ccc(N=C(OC)c3ccccc3)cc2)cc1)c1ccccc1]

To 20 g (44 mmol) of trimethyl orthobenzoate and 11.4 g (46 mmol) of 4,4-diaminodiphenyl sulfone in 75 ml of toluene was added a catalytic amount of a para-toluenesulfonic acid monohydrate, and the mixture was stirred for 5 hours under heating to reflux. Methanol, which was from reflux at the reaction system temperature of 100° C. or lower, was removed by the Dean-Stark apparatus. After confirming the completion of the reaction by TLC, ethyl acetate/water was added to carry out liquid separation. The organic phase was washed with water, and dried over magnesium sulfate. After distilling off the solvent, crystallization in an ethyl acetate/hexane mixed solvent was carried out to give 22 g (99%) of Exemplary Compound (42). The resulting Exemplary Compound was identified by ¹H-NMR.

### Synthesis Example 9

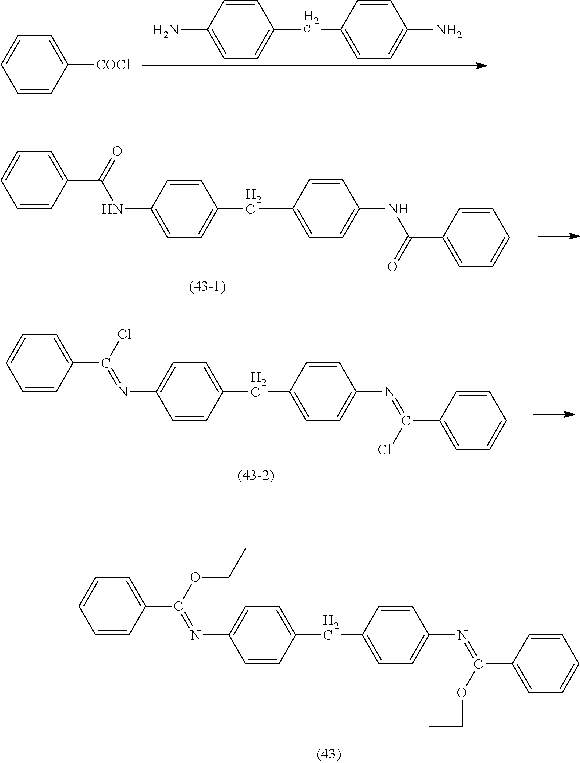

To a solution of 99 g (0.5 mol) of 4,4-diaminodiphenylmethane and 87 g (1.1 mol) of pyridine in 500 ml of N,N-dimethylacetamide was slowly added dropwise under ice-cooling 129 ml (1.1 mol) of benzoyl chloride. The temperature of the reaction system was allowed to warm to room temperature, and the completion of the reaction was confirmed by TLC. After 100 ml of methanol was added, the reaction solution was slowly added to 7 L of water to precipitate a solid. The solid was separated by filtration. The resulting solid was dispersed in methanol and filtered again. This operation was repeated twice. The resulting solid was dried to give 163 g (80%) of (43-1).

A suspension of 20 g (50 mmol) of (43-1) in 100 ml of thionyl chloride was stirred for 5 hours under heating to reflux. The complete dissolution of the reaction system was confirmed, followed by stirring for another 2 hours. Thereafter, thionyl chloride was distilled off and 300 ml of hexane was added to precipitate a solid. The solid was collected by filtration to give 22 g (quant.) of (43-2).

150 ml of a solution of 22 g (50 mol) of (43-2) in tetrahydrofuran was cooled to −5° C., and 37 g (110 mol) of a 20% solution of sodium ethoxide in ethanol was added dropwise thereto. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, purification was carried out by silica gel column chromatography using an ethyl acetate/hexane mixed solvent as an eluent. The solvent was distilled off to give 20 g (88%) of Exemplary Compound (43). The resulting Exemplary Compound was identified by ¹H-NMR.

### Synthesis Example 10

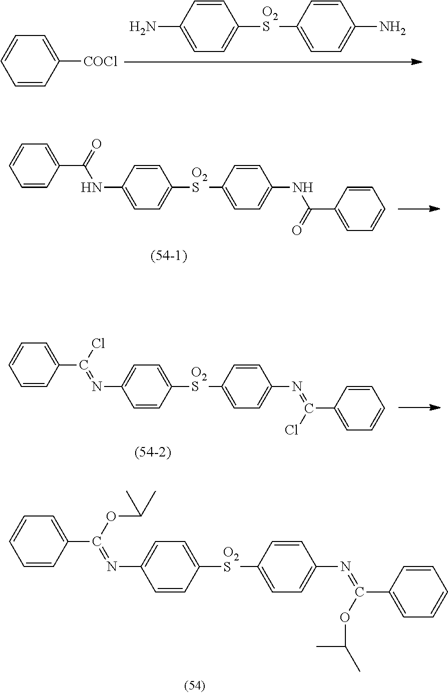 [SMILES: O=C(Cl)c1ccccc1 ; Nc1ccc(S(=O)(=O)c2ccc(N)cc2)cc1 ; O=C(Nc1ccc(S(=O)(=O)c2ccc(NC(=O)c3ccccc3)cc2)cc1)c1ccccc1 ; O=S(=O)(c1ccc(/N=C(\Cl)c2ccccc2)cc1)c1ccc(/N=C(\Cl)c2ccccc2)cc1 ; CC(C)O/C(=N\c1ccc(S(=O)(=O)c2ccc(/N=C(\OC(C)C)c3ccccc3)cc2)cc1)c1ccccc1]

To a solution of 124 g (0.5 mol) of 4,4-diaminodiphenylsulfone and 87 g (1.1 mol) of pyridine in 1 L of N,N-dimethylacetamide was slowly added dropwise 129 ml (1.1 mol) of benzoyl chloride under cooling. The temperature of the reaction system was allowed to warm to room temperature, followed by stirring for 5 hours. After 50 ml of methanol was added, the reaction solution was slowly added to 8 L of water to precipitate a solid. The solid was separated by filtration. The resulting solid was dispersed in water and filtered again. This operation was repeated twice. The resulting solid was dried to give 176 g (77%) of (54-1).

A suspension of 23 g (50 mmol) of (54-1) in 400 ml of thionyl chloride and a catalytic amount of N,N-dimethylformamide was stirred for 5 hours under heating to reflux. The complete dissolution of the reaction system was confirmed, followed by stirring for another 2 hours. Thereafter, thionyl chloride was distilled off to give 25 g (quant.) of (54-2).

4.4 g (0.11 mol) of sodium hydride (60%) was dispersed in hexane, and after allowing to stand, the supernatant hexane was removed by a Pasteur pipette. This operation was repeated twice. 100 ml of a tetrahydrofuran solution was added thereto. Under ice-cooling, 7 g (0.12 mol) of isopropanol was added dropwise thereto. Completion of bubble generation from the system was visually confirmed, and a solution of sodium isopropoxide in tetrahydrofuran was prepared.

150 ml of a solution of 25 g (50 mmol) of (54-2) in tetrahydrofuran was cooled to −5° C., and the prepared tetrahydrofuran solution of sodium isopropoxide was added dropwise thereto. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, crystallization in an ethyl acetate/hexane mixed solvent was carried out to give 23 g (86%) of Exemplary Compound (54). The resulting Exemplary Compound was identified by ¹H-NMR

### Synthesis Example 11

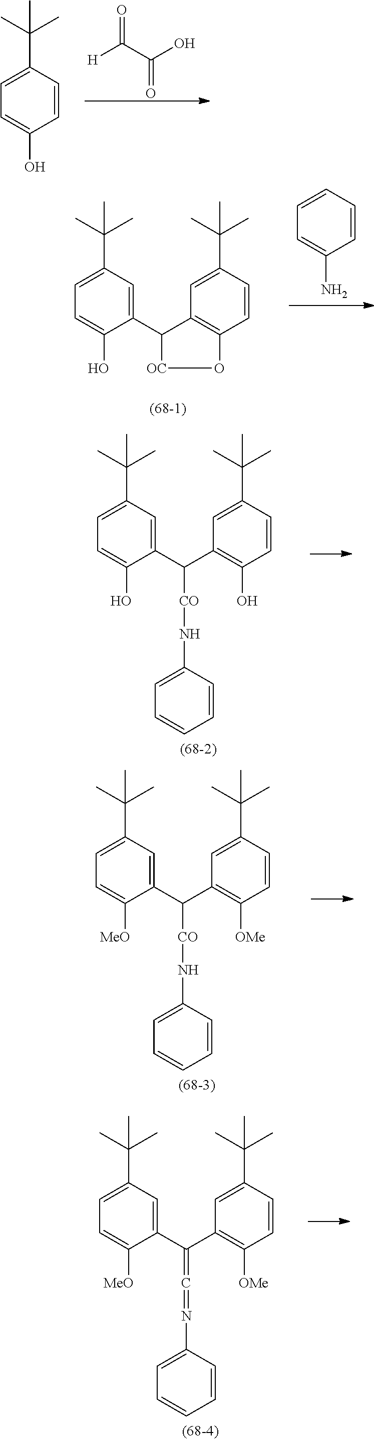

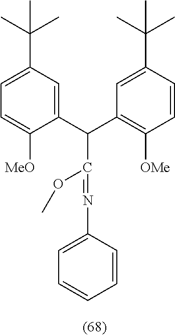 [SMILES: COC(=Nc1ccccc1)C(c1cc(C(C)(C)C)ccc1OC)c1cc(C(C)(C)C)ccc1OC]

To 45 g (0.3 mol) of 4-t-butylphenol and 13.8 g (0.15 mol) of glyoxylic acid monohydrate in 150 ml of hexane was added 2 ml of methanesulfonic acid, and the mixture was stirred for 1 hour under heating to reflux. Ethyl acetate/water was added to carry out liquid separation. The organic phase was washed with water, and dried over magnesium sulfate. After distilling off the solvent, crystallization in hexane was carried out to give 51 g (quant.) of (68-1).

20 g (59 mmol) of (68-1) and 53 g (0.57 mol) of aniline were stirred overnight at 70° C. Ethyl acetate/water was added to carry out liquid separation. The organic phase was washed with water, and dried over magnesium sulfate. After distilling off the solvent, crystallization in hexane was carried out to give 23 g (90%) of (68-2).

To a 200 ml solution of 20 g (46 mmol) of (68-2) and 8.7 ml (139 mmol) of methyl iodide in acetone was added 19.2 g of potassium carbonate, and the mixture was stirred at room temperature for 20 hours. The reaction system was filtered through Celite, and the filtrate was partitioned by adding ethyl acetate/water. The organic phase was washed with water and dried over magnesium sulfate. After distilling off the solvent, crystallization in an ethyl acetate/hexane mixed solvent was carried out to give 20 g (94%) of (68-3).

A solution of 15 g (32.6 mmol) of (68-3), 17.1 g (65.3 mmol) of triphenylphosphine, 6.3 ml (65.3 mmol) of carbon tetrachloride, and 9 ml (65.3 mmol) of triethylamine in 100 ml of chloroform was stirred for 8 hours under heating to reflux. The reaction system was subjected to reduced pressure. After distilling off chloroform, ethyl acetate was added and the solid was filtered through Celite. The filtrate was concentrated under reduced pressure and purified by silica gel column chromatography using an ethyl acetate/hexane mixed solvent as an eluent. The solvent was distilled off to give 11 g (76%) of (68-4).

A catalytic amount of sulfuric acid was added to 100 ml of methanol, and 8 g (17.4 mmol) of (68-4) was added thereto. After confirming the completion of the reaction by TLC, ethyl acetate/water was added to carry out liquid separation. The organic phase was washed with water, and dried over magnesium sulfate. After distilling off the solvent, purification was carried out by silica gel column chromatography using an ethyl acetate/hexane mixed solvent as an eluent. The solvent was distilled off to give 5 g (61%) of Exemplary Compound (68). The resulting Exemplary Compound was identified by ¹H-NMR.

### Synthesis Example 12

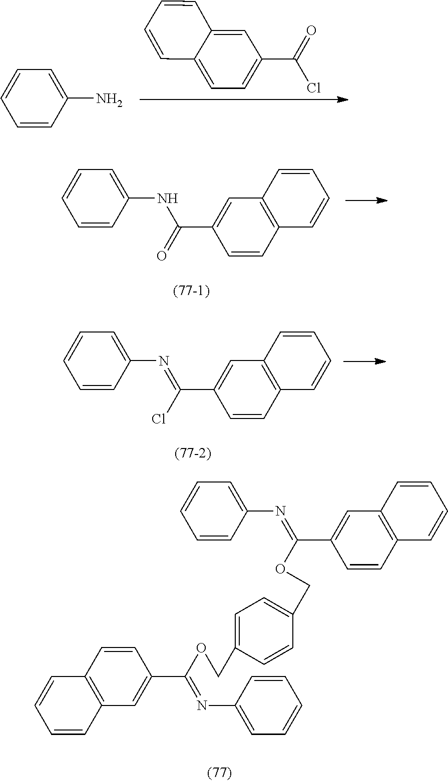 [SMILES: Nc1ccccc1 ; O=C(Cl)c1ccc2ccccc2c1 ; O=C(Nc1ccccc1)c1ccc2ccccc2c1 ; Cl/C(=N\c1ccccc1)c1ccc2ccccc2c1 ; c1ccc(/N=C(\OCc2ccc(CO/C(=N\c3ccccc3)c3ccc4ccccc4c3)cc2)c2ccc3ccccc3c2)cc1]

To a solution of 46.5 g (0.5 mol) of aniline and 44 g (0.6 mol) of pyridine in 300 ml of N,N-dimethylacetamide was slowly added under ice-cooling 95 g (0.5 mol) of 2-naphthalene carboxylic acid chloride. The temperature of the reaction system was allowed to warm to room temperature, and the completion of the reaction was confirmed by TLC. After 50 ml of methanol was added, the reaction solution was slowly added to 5 L of water to precipitate a solid. The solid was separated by filtration. The resulting solid was dispersed in methanol and filtered again. This operation was repeated twice. The resulting solid was dried to give 113 g (91%) of (77-1).

A suspension of 12.4 g (50 mmol) of (77-1) in 400 ml of thionyl chloride and a catalytic amount of N,N-dimethylformamide was stirred for 18 hours under heating to reflux. The complete dissolution of the reaction system was confirmed, followed by stirring for another 2 hours. Thereafter, thionyl chloride was distilled off to give 13.3 g (quant.) of (77-2).

2.0 g (50 mmol) of sodium hydride (60%) was dispersed in hexane, and after allowing to stand, the supernatant hexane was removed by a Pasteur pipette. This operation was repeated twice. 50 ml of a tetrahydrofuran solution was added thereto. Under ice-cooling, 3.5 g (25 mmol) of 1,4-benzene dimethanol was added. Completion of bubble generation from the system was visually confirmed, and a solution of 1,4-benzene dimethoxide in tetrahydrofuran was prepared.

150 ml of a solution of 13.2 g (50 mmol) of (77-2) in tetrahydrofuran was cooled to −5° C., and the prepared tetrahydrofuran solution of 1,4-benzene dimethoxide was added dropwise thereto. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, purification was carried out by silica gel column chromatography using an ethyl acetate/hexane mixed solvent as an eluent. The solvent was distilled off to give 9.2 g (62%) of Exemplary Compound (77). The resulting Exemplary Compound was identified by ¹H-NMR.

### Synthesis Example 13

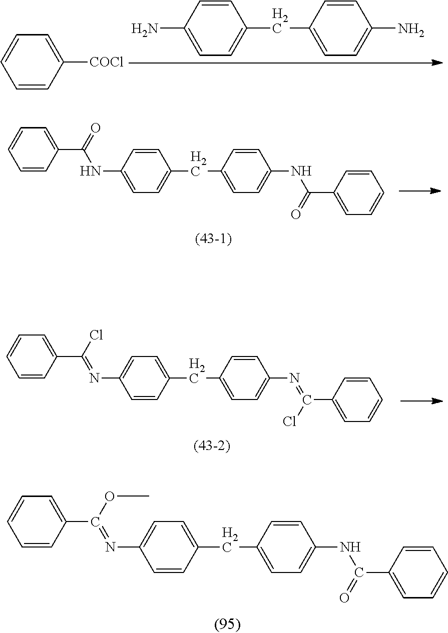 [SMILES: O=C(Cl)c1ccccc1 ; Nc1ccc(Cc2ccc(N)cc2)cc1 ; O=C(Nc1ccc(Cc2ccc(NC(=O)c3ccccc3)cc2)cc1)c1ccccc1 ; Cl/C(=N\c1ccc(Cc2ccc(/N=C(\Cl)c3ccccc3)cc2)cc1)c1ccccc1 ; CO/C(=N\c1ccc(Cc2ccc(NC(=O)c3ccccc3)cc2)cc1)c1ccccc1]

To a solution of 22 g (50 mmol) of (43-2) in 150 ml of tetrahydrofuran, which was synthesized in the same manner as in Synthesis Example 9, was added dropwise under ice-cooling 11 g (57 mmol) of a 28% solution of sodium methoxide in methanol. The temperature of the system was allowed to warm to room temperature, followed by stirring for 10 minutes, and ethyl acetate/water was added to carry out liquid separation. The organic phase was washed with water, and dried over magnesium sulfate. After distilling off the solvent, purification was carried out by silica gel column chromatography using an ethyl acetate/hexane mixed solvent as an eluent. The solvent was distilled off to give 16 g (77%) of Exemplary Compound (95). The resulting Exemplary Compound was identified by ¹H-NMR.

### Synthesis Example 14

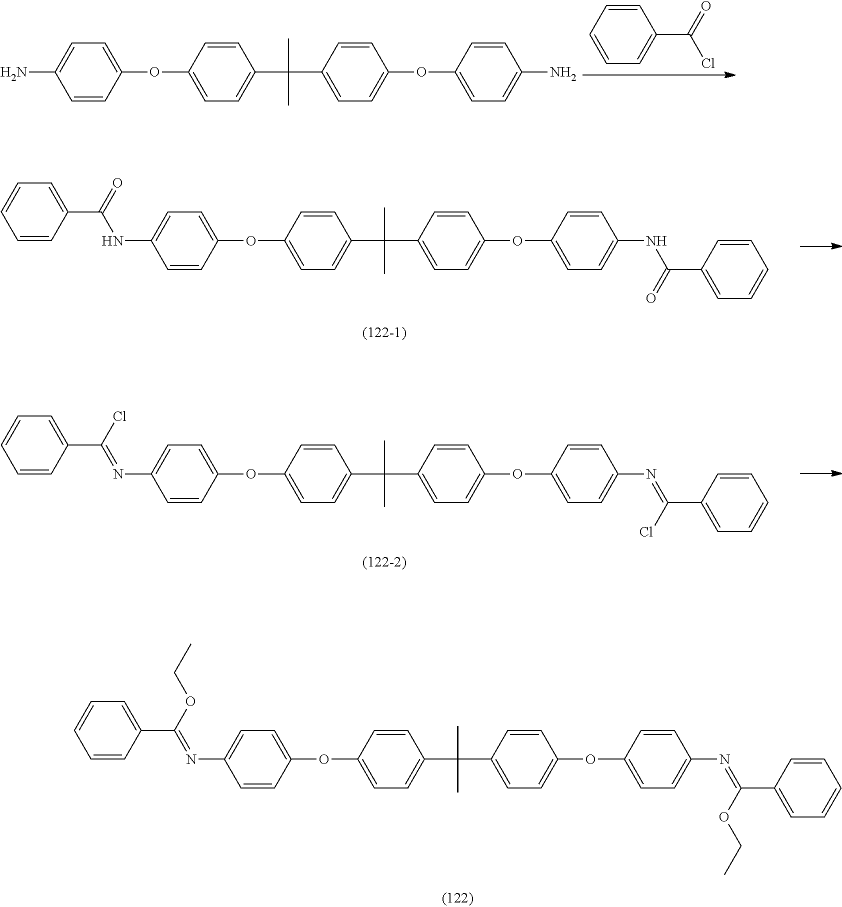 [SMILES: CC(C)(c1ccc(Oc2ccc(N)cc2)cc1)c1ccc(Oc2ccc(N)cc2)cc1 ; O=C(Cl)c1ccccc1 ; CC(C)(c1ccc(Oc2ccc(NC(=O)c3ccccc3)cc2)cc1)c1ccc(Oc2ccc(NC(=O)c3ccccc3)cc2)cc1 ; CC(C)(c1ccc(Oc2ccc(/N=C(\Cl)c3ccccc3)cc2)cc1)c1ccc(Oc2ccc(/N=C(\Cl)c3ccccc3)cc2)cc1 ; CCO/C(=N\c1ccc(Oc2ccc(C(C)(C)c3ccc(Oc4ccc(/N=C(\OCC)c5ccccc5)cc4)cc3)cc2)cc1)c1ccccc1]

Into a 5 L three-neck flask were charged 1.0 L of THF, 250 g (0.61 mol) of 2,2-bis [4-(4-aminophenoxy)phenyl]propane, and 148 g (0.146 mol) of triethylamine. Under ice-cooling, 205.5 g (0.1464 mol) of benzoyl chloride was slowly added dropwise thereto. After the dropwise addition, the temperature of the reaction system was allowed to warm to room temperature, followed by stirring for one hour and then the completion of the reaction was confirmed by TLC. 1.5 L of a 3.3% aqueous sodium hydroxide solution was slowly added dropwise to precipitate a solid. The solid was separated by filtration. The resulting solid was dispersed in 3 L of pure water and filtered again. The resulting solid was dried to give 372 g (99%) of (122-1).

Into a 2 L three-neck flask were charged 372 g (601.2 mol) of (122-1) and 1 L of thionyl chloride, and the mixture was stirred for 2 hours at 65° C. The completion of the reaction was confirmed by NMR. Then, thionyl chloride was distilled off to give 394 g (quant.) of (122-2).

Into a 5 L three-neck flask were charged 394 g (601.2 mol) of (122-2) and 1.2 L of THF, and 490.8 g (0.1443 mol) of 20% sodium ethoxide was slowly added dropwise thereto under ice-bath. After the dropwise addition, the temperature of the reaction system was allowed to warm to room temperature, followed by stirring for one hour and then the completion of the reaction was confirmed by TLC. 1.4 L of ethyl acetate was added, followed by liquid separation with 1.4 L of pure water and then liquid separation with 0.6 L of a 5% aqueous sodium chloride solution. After the organic layer was dried over magnesium sulfate, the solvent was distilled off to give a solid. 1.2 L of acetonitrile was added, followed by stirring for 2 hours at 40° C. and then for 2 hours at 10° C. The solid was filtered to give 334 g (84.6%) of Exemplary Compound (122). The resulting Exemplary Compound was identified by ¹H-NMR.

### Synthesis Example 15

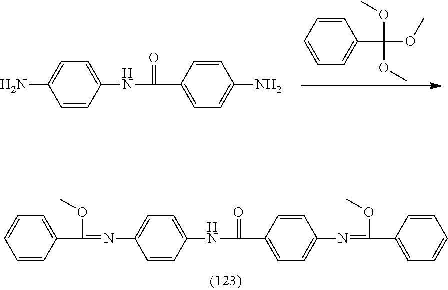 [SMILES: Nc1ccc(NC(=O)c2ccc(N)cc2)cc1 ; COC(OC)(OC)c1ccccc1 ; COC(=Nc1ccc(NC(=O)c2ccc(N=C(OC)c3ccccc3)cc2)cc1)c1ccccc1]

To 19.2 g (105.6 mmol) of trimethyl orthobenzoate and 10 g (44 mmol) of 4,4′-diaminobenzanilide in 45 ml of toluene was added a catalytic amount of a para-toluenesulfonic acid monohydrate, and the mixture was stirred for 5 hours under heating to reflux. Methanol, which was from reflux at the reaction system temperature of 100° C. or lower, was removed by the Dean-Stark apparatus. After confirming the completion of the reaction by TLC, the reaction system was allowed to cool to room temperature and methanol was added to carry out crystallization to give 18.5 g (91%) of Exemplary Compound (123). The resulting Exemplary Compound was identified by ¹H-NMR.

### Synthesis Example 16

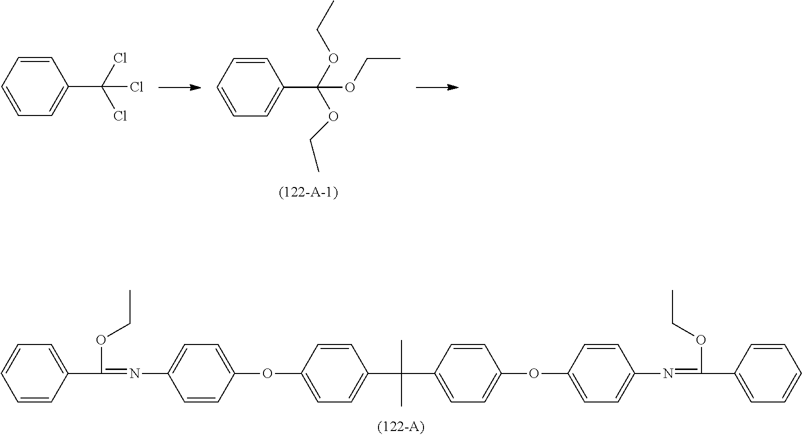 [SMILES: ClC(Cl)(Cl)c1ccccc1 ; CCOC(OCC)(OCC)c1ccccc1 ; CCOC(=Nc1ccc(Oc2ccc(C(C)(C)c3ccc(Oc4ccc(N=C(OCC)c5ccccc5)cc4)cc3)cc2)cc1)c1ccccc1]

Into a 5 L three-neck flask were charged 328 g (1.68 mol) of trichloromethyl benzene and 1867 g (5.487 mol) of a 20% solution of sodium ethoxide in ethanol. The mixture was stirred for 27 hours under heating to reflux, allowed to cool to room temperature and then filtered. The resulting solution was distilled off to give 601 g (2.681 mol) (80%) of (122-A-1). The resulting compound was identified by ¹H-NMR.

Into a 5 L three-neck flask were charged 600 g (2.80 mol) of (122-A-1), 498 g (1.28 mol) of 2,2-bis[4-(4-aminophenoxy)phenyl]propane, 480 mL of toluene, and 0.24 g (2.5 mmol) of methanesulfonic acid, followed by stirring for 2 hours under heating to reflux. Ethanol, which was from reflux at the reaction system temperature of 100° C. or lower, was removed by the Dean-Stark apparatus. After confirming the completion of the reaction by TLC, the reaction system was allowed to cool to room temperature and methanol was added to carry out crystallization to give 777 g (95%) of Exemplary Compound (122-A). The resulting Exemplary Compound was identified by ¹H-NMR.

### Example 1

### 1. Preparation of Saturated Polyester Resin

—Step (A)—

4.7 tons of high-purity terephthalic acid and 1.8 tons of ethylene glycol were mixed for 90 minutes to form a slurry, and the slurry was continuously supplied to a first esterification reaction tank at a flow rate of 3800 kg/h. Subsequently, an ethylene glycol solution of a citric acid chelated titanium complex (“VERTEC AC-420”, manufactured by Johnson Matthey Japan G.K.) having Ti metal coordinated with citric acid was continuously supplied to the first esterification reaction tank, and a reaction was carried out at a temperature inside the reaction tank of 250° C. and for an average retention time of about 4.4 hours with stirring, thereby obtaining an oligomer. At this time, the citric acid chelated titanium complex was continuously added such that the amount added of Ti was 9 ppm in terms of elements. The carboxylic acid value of the oligomer obtained was 500 eq/ton.

The obtained oligomer was transferred to a second esterification reaction tank, and with stirring, the reaction product was allowed to react at a temperature inside the reaction tank of 250° C. for an average retention time of 1.2 hours to obtain an oligomer having a carboxylic acid value of 180 eq/ton. The inside of the second esterification reaction tank was divided into three zones ranging a first zone to a third zone. At a second zone, an ethylene glycol solution of magnesium acetate was continuously supplied in a manner that the amount added of Mg was 75 ppm in terms of elements. After that, at a third zone, an ethylene glycol solution of trimethyl phosphate was continuously supplied in a manner that the amount added of P was 65 ppm in terms of elements. Moreover, the ethylene glycol solution of trimethyl phosphate was prepared by adding a trimethyl phosphate solution at 25° C. to an ethylene glycol solution at 25° C., followed by stirring at 25° C. for 2 hours (content of phosphorous compounds in the solution: 3.8% by mass). Thus, an esterification reaction product was obtained.

—Step (B)—

The esterification reaction product obtained in Step (A) was continuously supplied to a first polycondensation reaction tank. Subsequently, polycondensation (transesterification reaction) was carried out with stirring the esterification reaction product at a reaction temperature of 270° C. and a pressure inside the reaction tank of 20 torr (2.67×10⁻³ MPa) for an average retention time of about 1.8 hours.

Then, the obtained reaction product was transferred from the first polycondensation reaction tank to a second polycondensation reaction tank. Thereafter, in the second polycondensation reaction tank, the reaction product was subjected to a reaction (transesterification reaction) with stirring under the conditions of a temperature inside the reaction tank of 276□ and a pressure inside the reaction tank of 5 torr (6.67×10⁻⁴ MPa) for a retention time of about 1.2 hours.

Subsequently, the reaction product obtained by the transesterification reaction was transferred from the second polycondensation reaction tank to a third polycondensation reaction tank, and in this reaction tank, a reaction (transesterification reaction) was carried out with stirring under the conditions of a temperature inside the reaction tank of 276° C. and a pressure inside the reaction tank of 1.5 torr (2.0×10⁻⁴ MPa) for a retention time of 1.5 hours to obtain a reaction product (polyethylene terephthalate (PET)) having a carboxylic acid value of 22 eq/ton and an intrinsic viscosity (IV) of 0.65 dl/g.

—Preparation of Resin Pellet—

The obtained PET was put into a hopper of a twin-screw kneading extruder having a diameter of 50 mm using a main feeder, and Exemplary Compound 7 of the present invention was put into a subfeeder, followed by melting and extrusion at 280° C. The extruded molten material (melt) was withdrawn as a strand and then formed into a pellet.

—Performance Evaluation of Polyester Resin—

(Wet Heat Resistance (PCT Test))

Hydrolysis resistance was evaluated in terms of a carboxylic acid value of the above-mentioned pellet. The evaluation of the carboxylic acid value was made based on the value after a wet heat treatment. The wet heat treatment was carried out by subjecting the pellet to a storage treatment (heat treatment) for 105 hours under the conditions of 120° C. and relative humidity of 100%. The obtained results are shown in Table 2 below. The evaluation result of B grade or higher is preferable, and the evaluation result of A grade or higher is more preferable.

AA: The carboxylic acid value was 50 eq/ton or less

A: The carboxylic acid value was greater than 50 eq/ton and less than or equal to 100 eq/ton.

B: The carboxylic acid value was greater than 100 eq/ton and less than or equal to 200 eq/ton.

C: The carboxylic acid value was greater than 200 eq/ton.

### Examples 2 to 23

A polyester pellet of each of Examples was produced in the same manner as in Example 1, except that the type and amount of the iminoether compound were changed to those described in Table 1 below.

### Comparative Examples 1 to 3

A polyester pellet of each of Comparative Examples 1 to 3 was produced in the same manner as in Example 1, except that the type and amount of the iminoether compound were changed to those described in Table 1 below. Furthermore, the following Comparative Compound (1) poly(1,3,5-triisopropylphenylene-2,4-carbodiimide) (molecular weight: about 10,000) was used in Comparative Example 2, and the following Comparative Compound (2) 2,2′-(1,3-phenylene)bis(2-oxazoline) was used in Comparative Example 3.

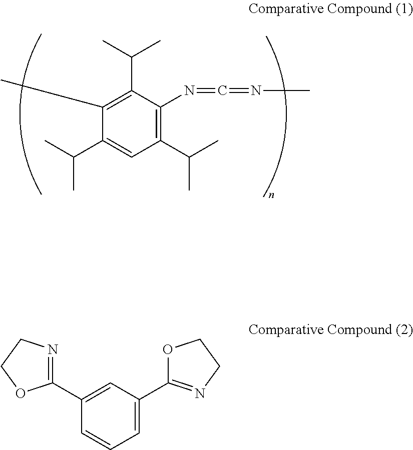

TABLE 1

| | Iminoether compound | | | | |
|---|---|---|---|---|---|
| | | Amount (% | | | |
| | | by mass) | Polyester resin | | |
| | | with | Carboxylic | Performance Evaluation | |
| | | respect to | acid value | Wet heat | |
| | Type | polyester | (eq/ton) | resistance | Production step |
| Example 1 | Exemplary | 0.4 | 12 | B | |
| | Compound (7) | | | | |
| Example 2 | Exemplary | 1 | 8 | A | |
| | Compound (7) | | | | |
| Example 3 | Exemplary | 2 | 5 | A | |
| | Compound (7) | | | | |
| Example 4 | Exemplary | 0.4 | 15 | B | |
| | Compound | | | | |
| | (17) | | | | |
| Example 5 | Exemplary | 1 | 9 | A | |
| | Compound | | | | |
| | (17) | | | | |
| Example 6 | Exemplary | 2 | 5 | A | |
| | Compound | | | | |
| | (17) | | | | |
| Example 7 | Exemplary | 1 | 8 | A | |
| | Compound | | | | |
| | (54) | | | | |
| Example 8 | Exemplary | 1 | 7 | A | |
| | Compound | | | | |
| | (13) | | | | |
| Example 9 | Exemplary | 1 | 8 | A | |
| | Compound | | | | |
| | (22) | | | | |
| Example | Exemplary | 1 | 7 | A | |
| 10 | Compound | | | | |
| | (36) | | | | |
| Example | Exemplary | 1 | 8 | A | |
| 11 | Compound | | | | |
| | (39) | | | | |
| Example | Exemplary | 1 | 8 | A | |
| 12 | Compound | | | | |
| | (68) | | | | |
| Example | Exemplary | 1 | 8 | A | |
| 13 | Compound | | | | |
| | (77) | | | | |
| Example | Exemplary | 1 | 8 | A | |
| 14 | Compound | | | | |
| | (95) | | | | |
| Example | Exemplary | 0.4 | 11 | A | |
| 15 | Compound | | | | |
| | (122) | | | | |
| Example | Exemplary | 1 | 7 | A | |
| 16 | Compound | | | | |
| | (122) | | | | |
| Example | Exemplary | 2 | 4 | A | |
| 17 | Compound | | | | |
| | (122) | | | | |
| Example | Exemplary | 0.4 | 10 | A | |
| 18 | Compound | | | | |
| | (123) | | | | |
| Example | Exemplary | 1 | 6 | A | |
| 19 | Compound | | | | |
| | (123) | | | | |
| Example | Exemplary | 2 | 4 | AA | |
| 20 | Compound | | | | |
| | (123) | | | | |
| Example | Exemplary | 0.4 | 10 | A | |
| 21 | Compound | | | | |
| | (122-A) | | | | |
| Example | Exemplary | 1 | 6 | A | |
| 22 | Compound | | | | |
| | (122-A) | | | | |
| Example | Exemplary | 2 | 4 | AA | |
| 23 | Compound | | | | |
| | (122-A) | | | | |
| Comparative | — | 0 | 22 | C | |
| Example 1 | | | | | |
| Comparative | Comparative | 1 | 5 | A | Irritant white smoke |
| Example 2 | Compound (1) | | | | occurred in |
| | | | | | production of pellet |
| Comparative | Comparative | 1 | 7 | B | Irritant white smoke |
| Example 3 | Compound (2) | | | | occurred in |
| | | | | | production of pellet |

As shown in Table 1 above, use of the iminoether compound represented by General Formula (1) employed in each of Examples can suppress the occurrence of irritant gases at the time of kneading, and therefore the obtained polyester pellet of each of Examples exhibits a low carboxylic acid value and excellent hydrolysis resistance even after the wet heat treatment.

### Example 24

### 2. Fabrication and Evaluation of Polyester Film

—Extrusion Molding (Synthesis Step/Film Forming Step)—

The PET obtained in step (B) was put into a hopper of double-screw kneading extruder having a diameter of 50 mm using a main feeder, and Exemplary Compound 7 of the present invention was put into a subfeeder, followed by melting and extrusion at 280° C. The extruded molten material (melt) was passed through a gear pump and a filter (pore diameter of 20 μm), and extruded from a die to a cooling roll at 20° C., whereby an amorphous sheet was obtained. Moreover, the extruded melt was adhered to the cooling roll using an electrostatic application method.

—Stretching (Biaxial Stretching Step)—

An unstretched film which was extruded onto the cooling roll and solidified was subjected to sequential biaxial stretching by the following method, whereby a polyester film having a thickness of 175 μm was obtained.

<Stretching Method>

(a) Longitudinal Stretching

The unstretched film is passed through between two pairs of nip rolls having different peripheral speed, and by this, the unstretched film was stretched in the longitudinal direction (transport direction). Moreover, the stretching was carried out at a preheating temperature of 90° C., a stretching temperature of 90° C., a stretching ratio of 3.5 times, and a stretching speed of 3,000%/sec.

(b) Transverse Stretching

The longitudinally stretched film was transversely stretched under the following conditions using a tenter.

<Conditions>
Preheating temperature: 100° C.
Stretching temperature: 110° C.
Stretching ratio: 4.2 times
Stretching speed: 70%/sec

—Heat Fixing and Thermal Relaxation—

Subsequently, the stretched film after finishing the longitudinal stretching and transverse stretching was heat-fixed under the following conditions. Furthermore, after heat fixing, the tenter width was shorten and thermal relaxation was carried out under the following conditions.

<Heat Fixing Conditions>
Heat fixing temperature: 198° C.
Heat fixing time: 2 seconds

<Thermal Relaxation Conditions>
Thermal relaxation temperature: 195° C.
Thermal relaxation ratio: 5%

—Winding—

After the heat fixing and the thermal relaxation, both ends of the polyester film were trimmed by 10 cm. Thereafter, both ends of the polyester film were subjected to an extrusion processing (knurling) at a width of 10 mm, followed by winding at a tension of 25 kg/m. Moreover, the width was 1.5 m, and the winding length was 2,000 m.

In the above manner, the polyester film of Example 24 was fabricated. The obtained polyester film was subjected to the following evaluation. The evaluation results are shown in Table 2 below.

—Process Evaluation—

(Gas)

Sensory evaluation of the smoke and smell generated from a die of a double-screw extruder was carried out, and the volatilization was evaluated based on the following criteria. The obtained results are shown in Table 2 below.

<Criteria>

A: There was no occurrence of smoke and smell.

B: There was no occurrence of smoke, but there was an occurrence of smell.

C: There was an occurrence of smoke and smell.

—Performance Evaluation of Polyester Film—

(Wet Heat Resistance (PCT Test))

Hydrolysis resistance was evaluated by a half-life period of a retention rate of elongation at break. The half-life period of a retention rate of elongation at break was evaluated by subjecting the polyester film obtained in Example 24 to a storage treatment (heat treatment) under the conditions of 120° C. and relative humidity of 100% and measuring the storage time when elongation at break (%) shown by the polyester film after storage becomes 50% of elongation at break (%) shown by the polyester film before storage. The obtained results are shown in Table 2 below.

AA: The half-life period of elongation at break was 180 hours or greater.

A: The half-life period of elongation at break was 160 hours or greater and less than 180 hours.

B: The half-life period of elongation at break was 130 hours or greater and less than 160 hours.

C: The half-life period of elongation at break was less than 130 hours.

It shows that the hydrolysis resistance of the polyester film is superior as the half-life period of a retention rate of elongation at break is longer. That is, in the polyester film of the present invention, the half-life period of elongation at break before and after the storage treatment under the conditions of 120° C. and relative humidity of 100% is preferably 130 hours or greater, more preferably 160 hours or greater and even more preferably 180 hours or greater.

(Volatile Component)

For the obtained polyester film, the amount of volatile components in the film was measured by gas chromatography (trade name P&T-GC/MS, manufactured by JASCO Corporation) according to the following criteria, and evaluation was carried out based on the following criteria. The obtained results are shown in Table 2 below.

<Conditions>

The obtained polyester film was heated at 280° C. for 10 minutes, and generated gas was detected.

<Criteria>

A: There was no occurrence of smoke and smell.

B: There was no occurrence of smoke, but there was an occurrence of smell.

C: There was an occurrence of smoke and smell.

(Film Surface State)

For the obtained polyester film, the film surface state was evaluated according to the following criteria.

The obtained results are shown in Table 2 below.

<Criteria>

A: When the film was visually observed, there was no unevenness on the film surface.

B: When the film was visually observed, there was unevenness on the film surface.

<Color Tone>

The obtained polyester film was visually observed and evaluated according to the following criteria.

A: Yellow tone was equivalent to that of Comparative Example (1), thus showing good results.

B: Yellow tone was greater than that of Comparative Example (1), thus showing inadequate results.

### Examples 25 to 46 and Comparative Examples 4 to 6

A polyester film of each of Examples and Comparative Examples was produced in the same manner as in Example 24, except that the type and amount of the iminoether compound were changed to those described in Table 2 below.

In each of Examples and Comparative Examples, the evaluation was carried out in the same manner as in Example 24. The obtained results are shown in Table 2 below.

TABLE 2

| | Iminoether compound | | | Polyester film performance | | | |
|---|---|---|---|---|---|---|---|
| | | Amount (% by mass) | Manufacturability | Wet heat | Volatile | Film | Color |
| | Type | relative to polyester | Gas volatilization | resistance | component | surface state | tone |
| Example 24 | Exemplary Compound (7) | 0.4 | A | B | A | A | A |
| Example 25 | Exemplary Compound (7) | 1 | A | A | A | A | A |
| Example 26 | Exemplary Compound (7) | 2 | A | A | A | A | B |
| Example 27 | Exemplary Compound (17) | 0.4 | A | B | A | A | A |
| Example 28 | Exemplary Compound (17) | 1 | A | A | A | A | A |
| Example 29 | Exemplary Compound (17) | 2 | A | A | A | A | B |
| Example 30 | Exemplary Compound (54) | 1 | A | A | A | A | A |
| Example 31 | Exemplary Compound (13) | 1 | A | A | A | A | A |
| Example 32 | Exemplary Compound (22) | 1 | A | A | A | A | A |
| Example 33 | Exemplary Compound (36) | 1 | A | A | A | A | A |
| Example 34 | Exemplary Compound (39) | 1 | A | A | A | A | A |
| Example 35 | Exemplary Compound (68) | 1 | A | A | A | A | A |
| Example 36 | Exemplary Compound (77) | 1 | A | A | A | A | A |
| Example 37 | Exemplary Compound (95) | 1 | A | A | A | A | A |
| Example 38 | Exemplary Compound (122) | 0.4 | A | A | A | A | A |
| Example 39 | Exemplary Compound (122) | 1 | A | A | A | A | A |
| Example 40 | Exemplary Compound (122) | 2 | A | A | A | A | B |
| Example 41 | Exemplary Compound (123) | 0.4 | A | A | A | A | A |
| Example 42 | Exemplary Compound (123) | 1 | A | A | A | A | A |
| Example 43 | Exemplary Compound (123) | 2 | A | AA | A | A | A |
| Example 44 | Exemplary Compound (122-A) | 0.4 | A | A | A | A | A |
| Example 45 | Exemplary Compound (122-A) | 1 | A | A | A | A | A |
| Example 46 | Exemplary Compound (122-A) | 2 | A | AA | A | A | A |
| Comparative | — | 0 | A | C | A | A | A |
| Example 4 | | | | | | | |
| Comparative | Comparative Compound (1) | 1 | C | A | C | A | B |
| Example 5 | | | | | | | |
| Comparative | Comparative Compound (2) | 1 | B | B | B | B | A |
| Example 6 | | | | | | | |

As can be seen from Table 2 above, the use of the iminoether compound represented by General Formula (1) of the present invention used in each of Examples could suppress an occurrence of irritating gases at the time of film formation, and the obtained polyester film of each of Examples was excellent in hydrolysis resistance and exhibited a low content of volatile components in the film. Also, the polyester film obtained in each of Examples was favorable in surface state thereof. Furthermore, the polyester film of each of Examples also exhibited good wet heat resistance. In particular, higher wet heat resistance was obtained in Examples 43 and 46.

On the other hand, due to no addition of a terminal blocking agent, wet heat resistance was remarkably inferior in Comparative Example 4. In addition, Comparative Example 5 exhibited a high content of volatile components in the polyester film, which results from significant volatilization of irritant gases during the production process. Comparative Example 6 was confirmed to have poor surface state of the polyester film and show unevenness on the film surface.

Using the polyester pellet prepared in Examples 1 to 23, polyester films were fabricated in the same manner as in Example 24, except that Exemplary Compound 7 was not further added. The polyester films fabricated through pellet in this manner also showed the same satisfactory results as those of Examples 24 to 46.

### 3. Fabrication of Back Sheet for Solar Cell Module

A back sheet for a solar cell module was fabricated using the polyester film fabricated in Example 24. First, on one surface of the polyester film fabricated in Example 24, the following (i) reflective layer and (ii) readily adhesive layer were applied in this order by coating.

(i) Reflective Layer (Colored Layer)

All the components having the following composition were mixed and subjected to a dispersion treatment for 1 hour with a dyno-mill disperser, thereby preparing a pigment dispersion.

<Formulation of Pigment Dispersion>

| | |
|---|---|
| Titanium dioxide | 39.9 parts |
| (TIPAQUE R-780-2, manufactured by Ishihara Sangyo | |
| Kaisha, Ltd., 100% by mass of solid content) | |
| Polyvinyl alcohol | 8.0 parts |
| (PVA-105, manufactured by Kuraray Co., Ltd., | |
| 10% of solid content) | |
| Surfactant | 0.5 parts |
| (DEMOL EP, manufactured by Kao Corporation, 25% | |
| of solid content) | |
| Distilled water | 51.6 parts |

Then, using the obtained pigment dispersion, all the components having the following composition were mixed to prepare a coating liquid for forming a reflective layer.

<Formulation of Coating Liquid for Forming Reflective Layer>

| | |
|---|---|
| Pigment dispersion above | 71.4 parts |
| Polyacrylic resin water dispersion | 17.1 parts |
| (binder: JURYMER ET410, manufactured by HAYASHI | |
| PURE CHEMICAL IND., LTD., 30% by mass of solid | |
| content) | |
| Polyoxyalkylene alkyl ether | 2.7 parts |
| (NAROACTY CL95, manufactured by Sanyo Chemical | |
| Industries, Ltd., 1% by mass of solid content) | |
| Oxazoline compound (crosslinking agent) | 1.8 parts |
| (EPOCROS WS-700, manufactured by NIPPON | |
| SHOKUBAI Co., Ltd., 25% by mass of solid content) | |
| Distilled water | 7.0 parts |

The coating liquid for forming a reflective layer obtained above was coated on the polyester film of Example 24 by a bar coater, and dried at 180° C. for 1 minute, thereby forming a (i) reflective layer (white layer) having a titanium dioxide coating amount of 6.5 g/m².

(ii) Readily Adhesive Layer

All the components having the following composition were mixed to prepare a coating liquid for forming a readily adhesive layer. The coating liquid was coated to a binder coating amount of 0.09 g/m² onto the (i) reflective layer, and then dried at 180° C. for 1 minute to form (ii) a readily adhesive layer.

<Composition of Coating Liquid for Forming Readily Adhesive Layer>

| | |
|---|---|
| Polyolefin resin water dispersion | 5.2 parts |
| (carboxylic acid-containing binder: CHEMIPEARL S75N, | |
| manufactured by Mitsui chemicals, Inc., 24% by | |
| mass of solid content) | |
| Polyoxyalkylene alkyl ether | 7.8 parts |
| (NAROACTY CL95, manufactured by Sanyo Chemical | |
| Industries, Ltd., 1% by mass of solid content) | |
| Oxazoline compound | 0.8 parts |
| (EPOCROS WS-700, manufactured by NIPPON | |
| SHOKUBAI Co., Ltd., 25% by mass of solid content) | |
| Silica fine particle water dispersion | 2.9 parts |
| (AEROSIL OX-50, manufactured by Nippon Aerosil | |
| Co., Ltd., 10% by mass of solid content) | |
| Distilled water | 83.3 parts |

Next, on the surface side opposite to the side having (i) the reflective layer and (ii) the readily adhesive layer of the polyester film formed thereon, the following (iii) undercoat layer, (iv) barrier layer, and (v) antifouling layer were applied by coating successively from the polyester film side.

(iii) Undercoat Layer

All the components having the following composition were mixed to prepare a coating liquid for forming an undercoat layer. This coating liquid was coated on the polyester film and dried at 180° C. for 1 minute to form an undercoat layer (dried coating amount: about 0.1 g/m²).

<Composition of Coating Liquid for Forming Undercoat Layer>

| | |
|---|---|
| Polyester resin | 1.7 parts |
| (VYLONAL MD-1200, manufactured by TOYOBO | |
| Co., Ltd., 17% by mass of solid content) | |
| Polyester resin | 3.8 parts |
| (sulfonic acid-containing binder: PESRESIN | |
| A-520, manufactured by TAKAMATSU OIL & FAT Co., | |
| Ltd. 30% by mass of solid content) | |
| Polyoxyalkylene alkyl ether | 1.5 parts |
| (NAROACTY CL95, manufactured by Sanyo Chemical | |
| Industries, Ltd., 1% by mass of solid content) | |
| Carbodiimide compound | 1.3 parts |
| (CARBODILITE V-02-L2, manufactured by Nisshinbo | |
| Holdings, Inc., 10% by mass of solid content) | |
| Distilled water | 91.7 parts |

(iv) Barrier Layer

Subsequently, on the surface of thus formed undercoat layer, an 800 angstroms thick vapor-deposited film of silicon oxide was formed under the following vapor deposition conditions. The film served as a barrier layer.

<Vapor Deposition Conditions>
Reactive gas mixing ratio (unit: slm): hexamethyldisiloxane/oxygen gas/helium=1/10/10
Vacuum degree inside vacuum chamber: 5.0×10⁻⁶ mbar
Vacuum degree inside vapor deposition chamber: 6.0×10⁻² mbar
Electric power supplied to cooling and electrode drums: 20 kW
Film transport speed: 80 m/minute

(v) Antifouling Layer

As shown below, coating liquids for forming a first antifouling layer and a second antifouling layer were prepared. The coating liquid for forming the first antifouling layer and the coating liquid for forming the second antifouling layer were coated in this order on the barrier layer, so that an antifouling layer having a bi-layer structure was applied by coating.

<First Antifouling Layer>

—Preparation of Coating Liquid for Forming First Antifouling Layer—

The components having the following composition were mixed to prepare a coating liquid for forming the first antifouling layer.

<Composition of Coating Liquid>

| | |
|---|---|
| CERANATE WSA1070 (manufactured by DIC Corporation) | 45.9 parts |
| Oxazoline compound (crosslinking agent) | 7.7 parts |
| (EPOCROS WS-700, manufactured by NIPPON SHOKUBAI | |
| Co., Ltd., 25% by mass of solid content) | |
| Polyoxyalkylene alkyl ether | 2.0 parts |
| (NAROACTY CL95, manufactured by Sanyo Chemical | |
| Industries, Ltd., 1% by mass of solid content) | |
| Pigment dispersion used for the reflective layer | 33.0 parts |
| Distilled water | 11.4 parts |

—Formation of First Antifouling Layer—

The obtained coating liquid was coated on the barrier layer to a binder coating amount of 3.0 g/m², and dried at 180° C. for 1 minute to form the first antifouling layer.

—Preparation of Coating Liquid for Forming Second Antifouling Layer—

The components having the following composition were mixed to prepare a coating liquid for forming the second antifouling layer.

<Composition of Coating Liquid>

| | |
|---|---|
| Fluorobinder | 45.9 parts |
| (OBBLIGATO, manufactured by AGC Coat-tech Co., Ltd.) | |
| Oxazoline compound | 7.7 parts |
| (EPOCROS WS-700, manufactured by NIPPON SHOKUBAI | |
| Co., Ltd., 25% by mass of solid content) (crosslinking agent) | |
| Polyoxyalkylene alkyl ether | 2.0 parts |
| (NAROACTY CL95, manufactured by Sanyo Chemical | |
| Industries, Ltd., 1% by mass of solid content) | |
| Pigment dispersion prepared for the reflective layer | 33.0 parts |
| Distilled water | 11.4 parts |

—Formation of Second Antifouling Layer—

The prepared coating liquid for forming the second antifouling layer was coated on the first antifouling layer formed on the barrier layer to a binder coating amount of 2.0 g/m², and dried at 180° C. for 1 minute to form the second antifouling layer.

As described above, the back sheet for a solar cell module was fabricated which has a reflective layer and a readily adhesive layer on one side of the polyester film and has an undercoat layer, a barrier layer and an antifouling layer on the other side.

[Production of Solar Cell]

Each of the back sheets for a solar cell module of the respective Examples produced as described above was adhered to a transparent filler so that the structure shown in FIG. 1 of JP2009-158952A was obtained, thereby producing a solar cell module. In the process, the back sheet was adhered so that the readily adhesive layer of the back sheet for a solar cell module of the respective Examples was in contact with the transparent filler embedding a solar cell device. The thus-produced solar cell module was confirmed to be capable of stably generating electricity over a long period of time.

According to the present invention, it is possible to suppress an occurrence of irritant gases in the production process of a polyester film containing a terminal blocking agent. Therefore, work safety during polyester film production can be enhanced. Furthermore, according to the present invention, even in the case where the terminal blocking agent is contained, it is possible to obtain a polyester film having a satisfactory film surface state without increasing the viscosity of the polyester resin, thus providing high industrial applicability.

What is claimed is:

1. A polyester resin composition comprising an iminoether compound represented by the following General Formula (1) and a polyester. 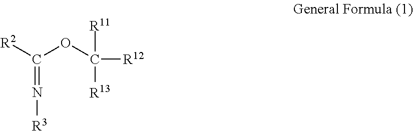
In General Formula (1), R² represents an alkyl group which may have a substituent, a cycloalkyl group which may have a substituent, an aryl group which may have a substituent or an alkoxy group which may have a substituent, R³ represents an alkyl group represented by the following General Formula (2) or an aryl group represented by the following General Formula (3), and R¹¹, R¹² and R¹³ each independently represent a hydrogen atom, an alkyl group which may have a substituent or an aryl group which may have a substituent; or alternatively, R², R³, R¹¹, R¹² and R¹³ may combine together to form a ring; provided that, in the case where R³ is represented by the following General Formula (2), a bond which is formed between at least one of R¹¹, R¹², or R¹³ and at least one of R³¹, R³², or R³³ is a bond in which the number of connecting atoms is 2 or more; 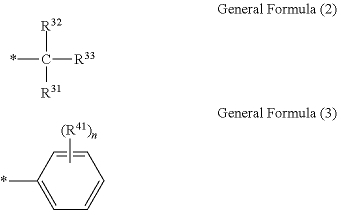
In General Formula (2), R³¹, R³² and R³³ each independently represent a hydrogen atom or a substituent; and R³¹, R³² and R³³ may combine together to form a ring; in General Formula (3), R⁴¹ represents a substituent, and in the case where a plurality of R⁴¹'s are present, they may be the same or different; n represents an integer of 0 to 5; and * in General Formulae (2) and (3) represents a site which is bonded to a nitrogen atom.

2. The polyester resin composition according to claim 1, wherein the iminoether compound is represented by the following General Formula (4); 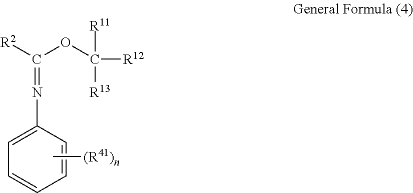
In General Formula (4), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; and n represents an integer of 0 to 5.

3. The polyester resin composition according to claim 1, wherein the iminoether compound is represented by the following General Formula (5); 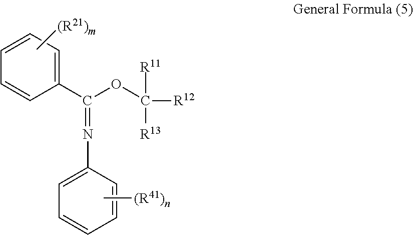
In General Formula (5), R¹¹, R¹² and R¹³ each independently represent a hydrogen atom, an alkyl group which may have a substituent or an aryl group which may have a substituent; R²¹ and R⁴¹ each independently represent a substituent; and in the case where a plurality of either of R²¹'s and R⁴¹'s are present, they may be the same or different; n represents an integer of 0 to 5, and m represents an integer of 0 to 5.

4. The polyester resin composition according to claim 1, wherein the iminoether compound is represented by the following General Formula (6); 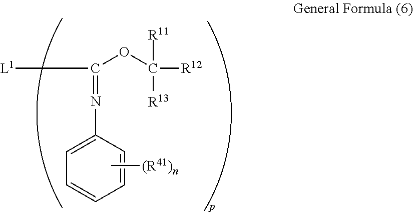
In General Formula (6), R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; n represents an integer of 0 to 5; p represents an integer of 2 to 4, and L¹ represents a p-valent group in which the end bonded to the carbon atom is an alkylene portion which may have a substituent, a cycloalkylene portion which may have a substituent, an arylene portion which may have a substituent, or an alkoxylene portion which may have a substituent.

5. The polyester resin composition according to claim 1, wherein the iminoether compound is represented by the following General Formula (7); 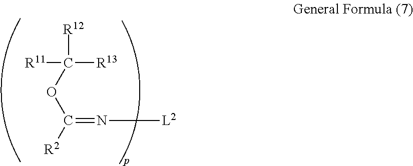
In General Formula (7), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; p represents an integer of 2 to 4, and L² represents a p-valent group in which the end bonded to the nitrogen atom is an arylene portion which may have a substituent, or a cycloalkylene portion which may have a substituent.

6. The polyester resin composition according to claim 1, wherein the iminoether compound is represented by the following General Formula (8); 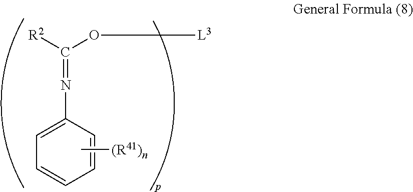
In General Formula (8), R² represents an alkyl group which may have a substituent, a cycloalkyl group which may have a substituent, an aryl group which may have a substituent or an alkoxy group which may have a substituent, R⁴¹ represents a substituent, and in the case where a plurality of R⁴¹'s are present, they may be the same or different; n represents an integer of 0 to 5; p represents an integer of 2 to 4, and L³ represents a p-valent group in which the end bonded to the oxygen atom is an alkylene portion; provided that some or all of hydrogen atoms in the alkylene portion of L³ may be substituted with an alkyl group which may have a substituent or an aryl group which may have a substituent.

7. The polyester resin composition according to claim 1, wherein the iminoether compound is prepared using an orthoester compound.

8. The polyester resin composition according to claim 1, wherein the composition includes 0.05 to 5% by mass of the iminoether compound with respect to the polyester.

9. The polyester resin composition according to claim 1, wherein a component derived from a carboxylic acid of the polyester is a component derived from an aromatic dibasic acid or an ester-forming derivative thereof.

10. An iminoether compound represented by the following General Formula (4); 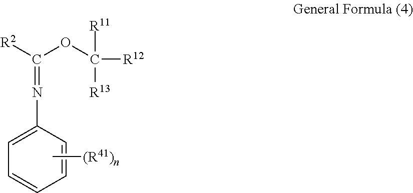
In General Formula (4), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; and n represents an integer of 0 to 5.

11. The iminoether compound according to claim 10, wherein the compound is represented by the following General Formula (5); 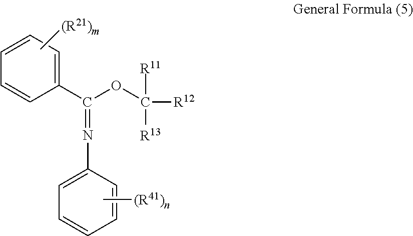
In General Formula (5), R¹² and R¹³ each independently represent a hydrogen atom, an alkyl group which may have a substituent or an aryl group which may have a substituent; R²¹ and R⁴¹ each independently represent a substituent; and in the case where a plurality of either of R²¹'s and R⁴¹'s are present, they may be the same or different; n represents an integer of 0 to 5, and m represents an integer of 0 to 5.

12. The iminoether compound according to claim 10, wherein the compound is represented by the following General Formula (6); 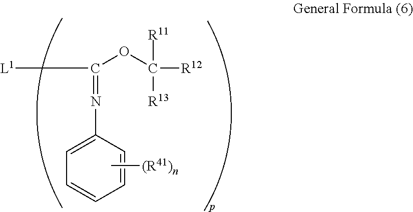
In General Formula (6), R¹¹, R¹² and R¹³ each independently represent a hydrogen atom, an alkyl group which may have a substituent or an aryl group which may have a substituent; R⁴¹ represents a substituent, and in the case where a plurality of R⁴¹'s are present, they may be the same or different; n represents an integer of 0 to 5; p represents an integer of 2 to 4, and L¹ represents a p-valent group in which the end bonded to the carbon atom is an alkylene portion which may have a substituent, a cycloalkylene portion which may have a substituent, an arylene portion which may have a substituent, or an alkoxylene portion which may have a substituent.

13. The iminoether compound according to claim 10, wherein the compound is represented by the following General Formula (7); 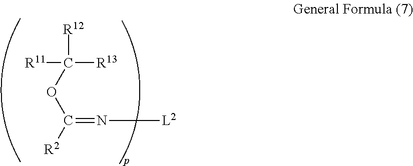
In General Formula (7), R² represents an alkyl group which may have a substituent, a cycloalkyl group which may have a substituent, an aryl group which may have a substituent or an alkoxy group which may have a substituent, R¹¹, R¹² and R¹³ each independently represent a hydrogen atom, an alkyl group which may have a substituent or an aryl group which may have a substituent; p represents an integer of 2 to 4, and L² represents a p-valent group in which the end bonded to the nitrogen atom is an arylene portion which may have a substituent.

14. The iminoether compound according to claim 10, wherein the compound is represented by the following General Formula (8); 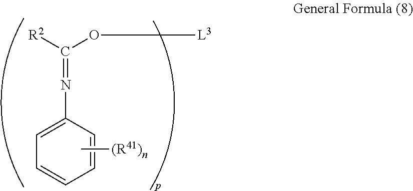
In General Formula (8), R² represents an alkyl group which may have a substituent, a cycloalkyl group which may have a substituent, an aryl group which may have a substituent or an alkoxy group which may have a substituent, R⁴¹ represents a substituent, and in the case where a plurality of R⁴¹'s are present, they may be the same or different; n represents an integer of 0 to 5; p represents an integer of 2 to 4, and L³ represents a p-valent group in which the end bonded to the oxygen atom is an alkylene portion; provided that some or all of hydrogen atoms in the alkylene portion of L³ may be substituted with an alkyl group which may have a substituent or an aryl group which may have a substituent.

15. The iminoether compound according to claim 10, wherein the compound is prepared using an orthoester compound.

16. A method for producing a carboxylic acid ester, comprising reacting the iminoether compound according to claim 10 with a compound having a carboxylic acid group at a temperature of 100 to 350° C. to produce a carboxylic acid ester.

17. A polyester film fabricated from the polyester resin composition according to claim 1.

18. The polyester film according to claim 17, wherein the film is biaxially stretched.

19. A back sheet for a solar cell module using the polyester film according to claim 17.

20. A solar cell module using the back sheet for a solar cell module according to claim 19.
